SEQUENCE LISTING (8 sequences)

SEQ ID NO: 1 - RNA, 23 nt, synthetic construct
  source 1..23: mol_type = other RNA
cccctggacacagagacttcatc

SEQ ID NO: 2 - RNA, 23 nt, synthetic construct
  source 1..23: mol_type = other RNA
ataccagcctcaaactcaccgac

SEQ ID NO: 3 - RNA, 20 nt, synthetic construct
  source 1..20: mol_type = other RNA
tctggtgatggtgtgaccca

SEQ ID NO: 4 - RNA, 20 nt, synthetic construct
  source 1..20: mol_type = other RNA
ggtgaagctgtagccacgct

SEQ ID NO: 5 - RNA, 23 nt, synthetic construct
  source 1..23: mol_type = other RNA
tggcagtgataggagattggctt

SEQ ID NO: 6 - RNA, 23 nt, synthetic construct
  source 1..23: mol_type = other RNA
agtaggacgtctcatggacccac

SEQ ID NO: 7 - RNA, 21 nt, synthetic construct
  source 1..21: mol_type = other RNA
atcgcatcacgcatcgctaaa

SEQ ID NO: 8 - RNA, 23 nt, synthetic construct
  source 1..23: mol_type = other RNA
ccgctgcgaaatacttcttctgt

(19) United States
(12) Patent Application Publication
(10) Pub. No.: US 2023/0321054 A1
(43) Pub. Date: Oct. 12, 2023

(54) COMPOSITIONS AND METHODS FOR CANCER AND TUMOR TREATMENT

(71) Applicant: Washington University, St. Louis, MO (US)

(72) Inventors: Gary Patti, St. Louis, MO (US); Fuad Naser, St. Louis, MO (US); Madelyn Jackstadt, St. Louis, MO (US)

(21) Appl. No.: 18/188,348

(22) Filed: Mar. 22, 2023

Related U.S. Application Data
(60) Provisional application No. 63/322,404, filed on Mar. 22, 2022.

(51) Int. Cl. A61P 35/00; A61K 31/4355

(52) U.S. Cl. CPC A61K 31/4355; A61P 35/00

(57) ABSTRACT

The present disclosure relates to the field of cancer and tumor therapeutics.

### GOVERNMENT SUPPORT

This invention was made with government support under ES028365 awarded by the National Institutes of Health. The government has certain rights in the invention.

### CROSS-REFERENCE TO RELATED APPLICATIONS

The present application claims the benefit of U.S. Provisional Pat. Application No. 63/322,404, entitled, “COMPOSITIONS AND METHODS FOR CANCER AND TUMOR TREATMENT” filed Mar. 22, 2022, the content of which is hereby incorporated by reference in its entirety.

### REFERENCE TO A SEQUENCE LISTING

This application contains a Sequence Listing that has been submitted in Extensible Markup Language (.xml) and is hereby incorporated by reference in its entirety. The XML copy, created on Mar. 22, 2023, is named 047563-756464.xml, and is 8.115 bytes in size.

### FIELD OF THE TECHNOLOGY

The present disclosure relates to the field of cancer and tumor therapeutics.

### BACKGROUND

The cell-intrinsic nature of tumor metabolism has become increasingly well characterized. The impact that tumors have on the systemic metabolism of a host, however, is less understood. Therefore, there is a need for development of model systems to study processes including metabolic crosstalk between tumors and distant, non-malignant tissues and how the nutritional burdens of a tumor impact the metabolic physiology of a host. Such model systems have the potential to improve understanding of systemic pathologies, such as cachexia, that are associated with cancer and help identify new targets for treatment of cancer.

### SUMMARY

In some aspects, the current disclosure encompasses a composition for treatment of cancer in a subject in need thereof, the composition comprising an inhibitor of alanine aminotransferase and at least a pharmaceutically acceptable excipient. In some aspects, the pharmaceutically acceptable excipient comprises a binder, a filler, a disintegrant, a lubricant, a glidant, a salt, a polymer, buffering agent, solvent, or a combination thereof. In some aspects of the current disclosure, the compositions as provided herein re effective in treatment of any cancer. In some aspects, the cancer is a melanoma. In some aspects, the subject is a vertebrate.. In some aspects, the composition is in the form of a tablet, a powder, a capsule, a liquid, or injectable. In some aspects, administration of the composition in the subject disrupts the tumor-liver alanine cycle.

In some aspects, the inhibitor of alanine aminotransferase is β-chloro-L-alanine or a pharmaceutically acceptable salt, derivative, variant or a functional analog thereof. In some aspects, the subject is a human. In some aspects, the composition is in a unit dose form wherein the β-Chloro-L-alanine or a pharmaceutically acceptable salt, derivative or analog thereof is present in an amount selected from a range of 10 mg to 2 grams.

In some aspects, the current disclosure also encompasses a method for treatment of a tumor or a cancer in a subject in need thereof, the method comprising administering to the subject an effective amount of a composition comprising an inhibitor of alanine aminotransferase and at least a pharmaceutically acceptable excipient. In some aspects, the inhibitor of alanine aminotransferase is β-chloro-L-alanine or a pharmaceutically acceptable salt, derivative or a functional analog thereof. In some aspects, the administration of the inhibitor disrupts the tumor-liver alanine cycle. In some aspects, the administration of the inhibitor results in a reduction of the dimensions of the tumor by at least about 10% to about 50%. In some aspects, the β-chloro-L-alanine or a pharmaceutically acceptable salt, derivative or analog thereof may be administered by an mode for example parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, and transdermal. In some aspects, the β-chloro-L-alanine is administered at a unit dose amount selected from a range of 10 mg to 2 grams. In some aspects of the method of treatment as disclosed herein, the cancer is a melanoma. In some aspects, the subject is a vertebrate for example a fish, a reptile, an amphibian or a mammal for example, a ovine, bovine, feline, porcine, rodent, primate or a human.

In some aspects, the current disclosure also encompasses a method of analysis of tumor metabolism in a laboratory animal tumor model, the method comprising: a) continuous administrating of an isotope tracers over a period of time through direct ingestion of fluids; b) obtaining a blood sample from the laboratory animal after step a); c) conducting a metabolomics analysis using an LC/MS; and comparing the hepatic gluconeogenesis flux in the laboratory animal tumor model with the hepatic gluconeogenesis flux of a healthy animal model without tumor. In some aspects, the laboratory animal is a zebra fish.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1K illustrates adding isotope tracers to adult zebrafish water achieves isotopic steady-state labeling. FIG. 1A is a schematic of the steady-state labeling workflow for LC/MS-based metabolomics. FIG. 1B depicts kinetics to achieve steady-state labeling for serum glucose from WT zebrafish in tank water containing 10 mM [U-13C] glucose. Values are mean ± SEM; n = 3-4 zebrafish per time point. FIG. 1C shows serum glucose levels in WT zebrafish over the course of 10 mM [U-13C] glucose labeling. Values are mean ± SEM; n = 3-4 zebrafish per time point. FIGS. 1D-1G illustrate fractional labeling, relative to serum M6 glucose, of the glycolytic intermediates glucose 6-phosphate (FIG. 1D), pyruvate (FIG. 1E), alanine (FIG. 1F), and lactate (FIG. 1G) in WT serum and liver between 18 and 24 hours. Data are mean ± SEM; n = 5 zebrafish per time point. FIGS. 1H-1K illustrate fractional labeling, relative to serum M6 glucose, of the metabolites a-ketoglutarate (FIG. 1H), succinate (FIG. 11), malate (FIG. 1J), and glutamine (FIG. 1K) in WT serum and liver between 18 and 24 hours. Data are mean ± SEM; n = 5 zebrafish per time point. n.d. = not detected; n.s. = not statistically significant according to a two-tailed paired t-test; G6-P, glucose 6-phosphate; Pyr, pyruvate; Ala, alanine; Lac, lactate; α-KG, α-ketoglutarate; Suc, succinate; Mal, malate; Gln, glutamine.

FIGS. 2A-2L shows the Optimization of [U-13C] glucose labeling workflow for metabolomics and glucose contribution to systemic metabolism in WT fish. FIG. 2A depicts distribution of serum volumes obtained when pipetting blood directly from a wound versus pooling blood by low-speed centrifugation following wound induction (see Online Methods for details). n = 10 zebrafish per condition. FIG. 2B illustrates distribution of circulating glucose levels when using tricaine and gradual cooling anesthesia. n = 7 zebrafish per condition. FIG. 2C displays kinetics to achieve steady-state labeling for serum glucose in zebrafish placed in 5 and 20 mM [U-13C] glucose. Values are mean ± SEM; n = 3-4 zebrafish per time point. FIG. 2D shows serum glucose levels in zebrafish over the course of 20 mM [U-13C] glucose labeling. Values are mean ± SEM; n = 3-4 zebrafish per time point. FIGS. 2E-2K illustrate comparison of zebrafish in standard facility water to zebrafish in 10 mM glucose water for 24 hours. The horizontal line denotes a p-value of 0.05. The two vertical lines denote a fold change of 2.0. No metabolites measured are above the designated thresholds in serum or any tissues. FIG. 2L shows normalized labeling of serum glucose, lactate, and glutamine shown together with normalized labeling of fumarate and malate from various tissues. Data were generated from WT zebrafish placed in [U-13C] glucose for 24 hours. Values are mean ± SEM; n = 5 zebrafish per condition.

FIGS. 3A-3E illustrates steady-state labeling of [U-13C] glutamine and contribution of glucose and glutamine to systemic metabolism. FIG. 3A shows kinetics to achieve steady-state labeling for serum glutamine in WT zebrafish placed in 5 mM [U-13C] glutamine. Values are mean ± SEM; n = 3-4 zebrafish per time point. FIG. 3B depicts relative glutamine levels in the serum of WT zebrafish before and after they were placed in 5 mM [U-13C] glutamine for 24 hours. Values are mean ± SEM; n = 3-4 zebrafish per time point. FIG. 3C displays fractional labeling, relative to serum M5 glutamine, of glutamate, α-ketoglutarate, malate, citrate, and proline in tissues from BRAF/p53 animals. Asparagine is not shown because no labeling was detected. Values are mean ± SEM; n =6 zebrafish per tissue. FIG. 3D shows fractional labeling, relative to serum M6 glucose, of lactate in tissues from BRAF/p53 zebrafish at isotopic steady state. Data are mean ± SEM; n = 10 zebrafish. FIG. 3E illustrates absolute enrichment of lactate (M3 isotopologue) over time in muscle from WT and BRAF/p53 fish following an intraperitoneal injection of [U-13C] glucose. Data are mean ± SEM; n = 3-4 zebrafish per condition for each time point. Statistically significant differences were assessed by a two-tailed paired t-test and annotated as follows: *p < 0.05, **p < 0.01, or n.s. = not significant. Glu, glutamate; a-KG, α-ketoglutarate; Mal, malate; Cit, citrate; Pro, proline.

FIGS. 4A-4G illustrates that Glucose is a major carbon source for melanoma metabolism in vivo. FIG. 4A is a schematic to illustrate the transformation of glutamine carbon to key intermediates in central carbon metabolism. FIG. 4B shows fractional labeling in tumor M5 glutamine, normalized to serum M5 glutamine, from a [U-13C] glutamine tracer. Values are mean ± SEM; n = 6 zebrafish. FIG. 4C is a schematic to illustrate the transformation of glucose carbon to key intermediates in central carbon metabolism. FIG. 4D illustrates comparison of the ¹³C-contribution from glucose and glutamine tracers to central carbon metabolites in melanoma. As expected, the isotopologues shown accounted for the majority of the labeling. Data shown here are presented as fold changes (i.e., the amount of labeling from [U-13C] glucose relative to the amount of labeling from [U-13C] glutamine, which is normalized to 1). Values are mean ± SEM; n = 6-10 zebrafish per condition. FIG. 4E depicts absolute enrichment of lactate (M3 isotopologue) over time in tissues from BRAF/p53 zebrafish following an intraperitoneal injection of [U-13C] glucose. Data are mean ± SEM; n = 3-4 zebrafish per time point. FIG. 4F shows Glucose uptake in tissues from BRAF/p53 zebrafish. Relative concentration of 2-deoxyglucose 6-phosphate (2-DG6-P) for tissues within a fish are normalized to the 2-DG6-P pool in its tumor. Values are mean ± SEM; n = 6 zebrafish per condition. FIG. 4G displays relative pool size of circulating glucose in WT and BRAF/p53 fish, normalized to the WT group. Values are mean ± SEM; n = 4-6 zebrafish per condition. Statistically significant differences were assessed by a two-tailed paired t-test and annotated as follows: *p < 0.05, **p < 0.01, ***p < 0.001, ****p < 0.0001, or n.s. = not significant. Lac, lactate; Ala, alanine; Cit, citrate; a-KG, α-ketoglutarate; Fum, fumarate; Mal, malate; Asp, aspartate.

FIGS. 5A-5J shows systems-level isotope tracing reveals elevated hepatic gluconeogenesis in BRAF/p53 fish, fueled by melanoma-derived alanine. FIG. 5A depicts heatmap of normalized labeling differences in various metabolites from [U-13C] glucose. Differences were calculated by comparing the normalized labeling of a tissue from BRAF/p53 fish to the normalized labeling of the same tissue from WT fish. Green indicates increased labeling and red indicates decreased labeling in metabolites from BRAF/p53 fish. Each data point represents the ratio of means from biological replicates. White boxes denote metabolites that did not incorporate ¹³C tracer. n = 5-10 zebrafish per condition. FIG. 5B depicts heatmap of metabolite pool-size differences in tissues from BRAF/p53 fish relative to tissues from WT fish. Green indicates larger pool size and red indicates smaller pool size of metabolites from BRAF/p53 fish. Each data point represents the ratio of means from biological replicates. n = 5-10 zebrafish per condition. FIG. 5C illustrates relative concentration of hepatic glycogen in WT and BRAF/p53 fish, normalized to the WT group. Values are mean ± SEM; n = 7 zebrafish per condition. FIG. 5D shows relative expression of the hepatic gluconeogenic enzymes glucose 6-phosphatase (G6Pase) and phosphoenolpyruvate carboxykinase (PEPCK) in BRAF/p53 fish normalized to WT expression. Values are mean ± SEM; n = 4-5 zebrafish per group. FIGS. 5E-5H display fractional labeling, relative to serum M6 glucose, of isotopologues for glucose (FIG. 5F), lactate (FIG. 5G), alanine (FIG. 5H), and pyruvate (FIG. 51) in serum and liver of WT and BRAF/p53 animals. Data are presented as fold changes relative to the WT group. Values are mean ± SEM; n = 5-10 zebrafish per condition. FIG. 51 shows fractional labeling, relative to serum M6 glucose, of alanine in tissues from BRAF/p53 animals. Data are presented as fold changes relative to alanine labeling in tumors. Values are mean ± SEM; n = 10 zebrafish per condition. FIG. 5J illustrates alanine uptake in tissues from BRAF/p53 zebrafish. Pool sizes of 2-aminoisobutyric acid (2-AIB) for tissues within a fish are normalized to the 2-AIB pool in its tumor. Values are mean ± SEM; n = 6 zebrafish per condition. Statistically significant differences were assessed by a two-tailed paired t-test and annotated as follows: *p < 0.05, **p < 0.01, or n.s. = not significant.

FIGS. 6A-6E illustrates comparison of hepatic glycogen, liver mass, and gluconeogenic activity between WT and BRAF/p53 zebrafish. FIG. 6A shows Periodic Acid-Schiff (PAS) staining of 5 µm liver sections from WT or BRAF/p53 fish. WT livers show abundant glycogen staining that is reduced in the livers of BRAF/p53 livers. Diastase digestion was used as a control to confirm glycogen staining. Scale bar = 50 µm. FIG. 6B dsiplays the relative mass of livers is decreased in melanoma-bearing animals compared to WT, consistent with reduced glycogen content. Relative liver mass was determined as the mass of the liver divided by the mass of the whole animal. n = 23-25 zebrafish per group. FIG. 6C depicts the relative mass of livers is decreased in melanoma-bearing animals compared to WT, even when corrected for tumor mass. To correct for tumor mass, the relative liver mass was determined as the mass of the liver divided by the mass of the whole animal after subtracting the mass of the tumors. n = 23-25 zebrafish per group. FIG. 6D is a schematic of metabolic transformations associated with hepatic gluconeogenesis. Pyruvate carboxylase (PC) activity transforms [U-¹³C] pyruvate into malate with three ¹³C labels. Oxaloacetate and malate are in equilibrium. M3 labeling in malate is indicative of gluconeogenic flux. FIG. 6E shows fractional labeling, relative to serum M6 glucose, of malate from liver of WT and BRAF/p53 fish. Data are normalized to the WT group. Values are mean ± SEM; n = 5-10 zebrafish per condition. Statistically significant differences were assessed by a two-tailed paired t-test (including a Welch’s correction for panels B and C), *p < 0.05, **p < 0.01, or ***p < 0.001.

FIGS. 7A-7I shows circulating BCAAs provide melanoma with a source of nitrogen for the alanine cycle, which can be impaired by vemurafenib. FIG. 7A shows concentration of ammonia excreted into tank water from WT and BRAF/p53 fish. Measurements were made 24 hours after water refreshing. Each group was housed separately. Values are mean ± SEM; n = 4 zebrafish per condition. FIG. 7B illustrates Fractional labeling, relative to M6 glucose, of M3 alanine and M3 glucose in BRAF/p53 zebrafish treated with EIPA or DMSO (Veh). Values are mean ± SEM; n = 10-14 zebrafish per condition. FIG. 7C displays Fractional labeling, relative to serum M1 BCAAs, of glutamate in tumor and muscle of BRAF/p53 fish. Serum M1 BCAAs was determined by averaging the values of M1 from isoleucine, leucine, and valine. Data are available in Data 8. Values are mean ± SEM; n = 5 zebrafish. FIG. 7D shows fractional labeling, relative to serum M1 BCAAs, of isoleucine, leucine, and valine in serum and tumor of BRAF/p53 fish. Values are mean ± SEM; n = 5 zebrafish per condition. FIG. 7E illustrates BCAT1 expression in zebrafish melanocytes and BRAF/p53 melanoma cells. Center line, median; box limits are first and third quartile; whiskers, range. FIG. 7F shows BCAT1 expression in human melanocytes and human melanoma. FIG. 7G is a schematic of when vemurafenib and [U-¹³C] glucose were administered to melanoma-bearing zebrafish. Red triangles denote the timing of vemurafenib injections. FIG. 7H illustrates fractional labeling, relative to serum M6 glucose, of the central carbon intermediates ribose 5-phosphate (left), glucose 6-phosphate (middle), and alanine (right) in tumors treated with either DMSO (Veh) or vemurafenib (Vem). Data are normalized to the vehicle-treated group. Values are mean ± SEM; n = 5-6 zebrafish per condition. FIG. 7I depicts fractional labeling, relative to serum M6 glucose, of gluconeogenesis-derived glucose (left) and alanine (right) in circulation and liver of vehicle-treated WT, vehicle-treated BRAF/p53, and vemurafenib-treated BRAF/p53 fish. Data are normalized to the vehicle-treated WT group. Values are mean ± SEM; n = 5-6 zebrafish per condition. Statistically significant differences were assessed by a two-tailed paired t-test (FIGS. 7A-7D, FIGS. 7H-7I) or a Wilcoxon rank sum test (FIGS. 7E-7F) and annotated as follows: *p < 0.05, **p < 0.01, ***p < 0.001 or n.s. = not significant. R5-P, ribose 5-phosphate; G6-P, glucose 6-phosphate; Ala, alanine; Glc, glucose.

FIGS. 8A-8D shows relative levels of circulating amino acids in zebrafish and effects of vemurafenib treatment on melanoma. FIG. 8A illustrates relative pool sizes of amino acids in serum of WT and BRAF/p53 zebrafish, normalized to isoleucine, the most abundant metabolite in serum. Data are available in Data 7. Values are mean ± SEM; n = 5-10 zebrafish per group. FIG. 8B shows representative images of a BRAF/p53 zebrafish before and after 4 consecutive days of vemurafenib intraperitoneal injections (100 mg/kg/day). FIG. 8C illustrates concentration of ammonia excreted into tank water from vehicle-treated BRAF/p53 zebrafish, BRAF/p53 zebrafish treated with vemurafenib for 4 consecutive days, and BRAF/p53 zebrafish treated with vemurafenib for 10 consecutive days. Measurements were made 24 hours after water refreshing. Measurements were made from animals in separate tanks. Values are mean ± SEM; n = 4 zebrafish per condition. FIG. 8D depicts fractional labeling, relative to serum M6 glucose, of a-ketoglutarate from tumors of vehicle-treated and vemurafenib-treated BRAF/p53 fish. Data are normalized to the vehicle-treated group. Values are mean ± SEM; n = 5-6 zebrafish per group. FIG. 8D also shows fractional labeling, relative to serum M6 glucose, of malate from tumors of vehicle-treated and vemurafenib-treated BRAF/p53 fish. Data are normalized to the vehicle-treated group. Values are mean ± SEM; n = 5-6 zebrafish per group. FIG. 8D additionally displays pool size of ribose 5-phosphate from melanoma in vehicle-treated BRAF/p53 zebrafish relative to vemurafenib-treated BRAF/p53 zebrafish. Data are normalized to the vehicle-treated group. Values are mean ± SEM; n = 5-6 zebrafish per group. Statistically significant differences were assessed by a two-tailed paired t-test and annotated as follows: *p < 0.05 or **p < 0.01. R5-P, ribose 5-phosphate; a-KG, a-ketoglutarate; Mal, malate.

FIGS. 9A-9I shows that vemurafenib does not lead to off-target metabolic effects in WT zebrafish and yields a comparable metabolic phenotype in BRAF/p53 zebrafish after 4 and 10 days of treatment. FIGS. 9A-9F illustrate fractional labeling, relative to serum M6 glucose, of glucose (FIG. 9A), lactate (FIG. 9B), alanine (FIG. 9C), citrate (FIG. 9D), a-ketoglutarate (FIG. 9E), and malate (FIG. 9F) in tissues of vehicle-treated or vemurafenib-treated WT fish. Animals were treated with vemurafenib for 4 days. Data are normalized to the vehicle-treated group. Values are mean ± SEM; n = 12 zebrafish per condition. FIG. 9G shows Representative images of two BRAF/p53 zebrafish before and after 10 days of consecutive vemurafenib intraperitoneal injections (100 mg/kg/day). FIG. 9H illustrates tumor regression over time as a result of daily vemurafenib treatment. Tumor size was measured by using digital calipers. Values are mean ± SEM; n = 3 animals per time point. FIG. 9I shows fractional labeling, relative to serum M6 glucose, of glucose in serum of vehicle-treated WT fish, vehicle-treated BRAF/p53 fish, and vemurafenib-treated BRAF/p53 fish. Animals were treated with vemurafenib for 4 or 10 consecutive days. Data are normalized to the vehicle-treated WT group, and labeling is normalized to the glucose pool size in serum. Values are mean ± SEM; n = 3-6 zebrafish per condition. Statistically significant differences were assessed by a two-tailed paired t-test and annotated as follows: *p < 0.05 or n.s. = not significant.

FIGS. 10A-10E shows that pharmacologically inhibiting ALT provides direct support for tumor-liver alanine cycling. FIG. 10A is a schematic to illustrate the transformation of BCAA carbon to key intermediates in central carbon metabolism. FIG. 10B illustrates fractional labeling, relative to serum [U-¹³C] BCAAs, of the central carbon intermediates a-ketoglutarate, glutamate, malate, and fumarate in tumors treated with either vehicle (Veh) or β-chloroalanine (ALTi). Data are normalized to the vehicle-treated group. Serum [U-¹³C] BCAAs was determined by averaging [U-¹³C] isoleucine, [U-¹³C] leucine, and [U-¹³C] valine. Values are mean ± SEM; n = 6-7 zebrafish per condition. FIG. 10C shows relative pool sizes of central carbon metabolites from BRAF/p53 animals treated with either vehicle (Veh) or β-chloroalanine (ALTi). Data are normalized to the vehicle-treated group. Values are mean ± SEM; n = 6-10 zebrafish per condition. FIG. 10D illustrates relative change from baseline in tumor volume after 10 days of β-chloroalanine (ALT inhibitor) treatment. Values are mean ± SEM; n = 9-10 tumors per condition. FIG. 10E depicts representative image of tumor regression in a BRAF/p53 fish after 10 days of β-chloroalanine treatment. Statistically significant differences were assessed by a two-tailed paired t-test and annotated as follows: *p < 0.05, **p < 0.01, ***p < 0.001, ****p < 0.0001, or n.s. = not significant.

FIGS. 11A-11B illustrates reduction in tumor volume in mouse with administration of gluconeogenesis inhibitor. FIG. 11A shows average tumor volume measured by IVIS imaging in mice with glioblastoma treated for 5 days with either vehicle or 50 mg/kg of a gluconeogenesis inhibitor. FIG. 11B shows representative bioluminescent images at day 6 after tumor induction in a vehicle treated control mouse or a mouse that received a gluconeogenesis inhibitor.

FIG. 12 illustrates that nonmalignant tissues alter their metabolism to support tumor growth and demonstrates that glucose-derived alanine is excreted from melanoma and transferred to the liver for gluconeogenesis.

The drawing figures do not limit the present inventive concept to the specific aspects disclosed and described herein. The drawings are not necessarily to scale, emphasis instead being placed on clearly illustrating principles of certain aspects of the present inventive concept.

### DETAILED DESCRIPTION

Provided herein are compositions and methods for treating various cancer and tumors. As described herein, the present disclosure provides compositions that reduce cancer or tumor growth by disrupting tumor metabolism as well as limiting liver-derived glucose for use by the cancer and/or tumor cells (e.g., non-limiting examples include melanoma, non-small cell lung carcinoma, nasopharyngeal carcinoma, ovarian cancer, breast cancer, hepatocellular carcinoma, chronic myeloid leukemia, and glioblastoma). The impact that tumors have on the systemic metabolism of a host is not well understood. The present disclosure shows melanomas consume ~15-times more glucose than other tissues measured. Interestingly, despite this pathological burden on glucose homeostasis, glucose levels are maintained in the circulation by a tumor-liver alanine cycle. Excretion of glucose-derived alanine from tumor or cancer cells provide both a source of carbon for hepatic gluconeogenesis and allow the tumor or cancer cell to remove excess nitrogen that results from activated branched-chain amino acid catabolism, which was found to be characteristic of melanoma.

Additional aspects and iterations of the disclosure are described below.

### Definitions

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

“About” is used to provide flexibility to a numerical range endpoint by providing that a given value may be “slightly above” or “slightly below” the endpoint without affecting the desired result. The term “about” in association with a numerical value means that the numerical value can vary plus or minus by 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. Those in need of treatment include those already with the disorder as well those prone to have disorder or those in whom disorder is to be prevented.

As used herein, “prevent” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount” or “therapeutically effective amount” refers to an amount sufficient to effect beneficial or desirable biological and/or clinical results.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some aspects, the subject can be a human. In other aspects, the subject can be a human in need of treating a cancer.

As used herein “cancer” may be one or more neoplasm or cancer. The neoplasm may be malignant or benign, the cancer may be primary or metastatic; the neoplasm or cancer may be early stage or late stage. Non-limiting examples of neoplasms or cancers include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing’s sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), enknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

As used herein, treatment of a cancer or tumor can comprise increased inhibition of cancer progression and/or metastases, inhibition of an increase in tumor volume, a reduction in tumor volume and/or growth, a reduction in tumor growth rate, an eradication of a tumor and/or cancer cell, or any combination thereof. In some aspects, the treatment can also prolong the survival of a subject, improve the prognosis and/or improve the quality of life of the subject.

As used herein “gluconeogenesis” refers to a metabolic pathway that results in the generation of glucose from certain non-carbohydrate carbon substrates or from non-hexose precursors, such as glycerol, lactate, pyruvate, or amino acids.

As used herein “accumulation” refers to the amount of a particular analyte (e.g., tumor or cancer cell) present in the sample. The amount may be a number, ratio, proportion, or a percentage of the analyte compared to the control sample or determined using a standard curve. The amount may be an absolute amount or a relative amount.

As used herein “control sample” or “control cell” can be procured from a healthy subject and/or a subject with cancer procured prior to the start of treatment (baseline). In some aspects, the control sample can comprise non-cancer cells. In some aspects, the non-cancer cells can be from the same tissue type as the cancer cells. For example, if the cancer cells are from breast cancer, then the non-cancer cells can be from healthy breast tissue. In some aspects, the control is a person or persons with similar characteristics to the subject with cancer. In some aspects, the control sample can be pooled sample.

As used herein, a biological sample may be of any biological tissue, fluid, or cell from the subject. The sample can be solid or fluid. The sample can be a heterogeneous cell population. Non-limiting examples of suitable biological samples include sputum, serum, blood, blood cells (e.g., white cells), a biopsy, urine, peritoneal fluid, pleural fluid, or cells derived therefrom. The biopsy can be a fine needle aspirate biopsy, a core needle biopsy, a vacuum assisted biopsy, an open surgical biopsy, a shave biopsy, a punch biopsy, an incisional biopsy, a curettage biopsy, or a deep shave biopsy. Biological samples may also include sections of tissues, such as frozen sections or formalin fixed sections taken for histological purposes. A sample can be a tumor tissue, tissue surrounding a tumor, or non-tumor tissue. Methods of collecting a biological sample from a subject are well known in the art.

As used herein, the following definitions shall apply unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March’s Advanced Organic Chemistry,” 5th Ed., Smith, M. B. and March, J., eds. John Wiley & Sons, New York: 2001, the entire contents of which are hereby incorporated by reference.

The present disclosure is partly based on the surprising discovery that tumor or cancer cells excrete alanine which provide a source of carbon for hepatic gluconeogenesis and allows the tumor or cancer cell to remove excess nitrogen that results from activated branched-chain amino acid catabolism.

### I. Inhibitors

In some aspects, the current disclosure provides an inhibitor of alanine aminotransferase (ALT).

In some aspects, an inhibitor of ALT comprises a chemical compound, antibody, or other biological inhibitor. In some aspects, an ALT inhibitor as described herein may decrease ALT activity in cells (e.g., tumor cell or cancer cells ) by at least 20%, e.g., 30%, 40%, 50%, 60%, 70%, 80%, 90%, 95% or above, as compared to baseline activity before administration of the inhibitor or a control cell. The decreased activity of ALT in the presence of at least one inhibitor can be determined by conventional methods, e.g., using colorimetric or fluorometric enzyme assay, or using a commercially available kit (e.g., Sigma-Aldrich MAK052-1 KT).

In some aspects, an inhibitor of ALT comprises a chemical compound. In some aspects, an inhibitor of ALT disrupts the tumor-liver alanine cycle. In some aspects, any chemical inhibitor that can block or inactivate alanine production or impair glucose uptake in a tumor cell or cancer cell of a subject is contemplated for use in the disclosed composition. Such inhibitors can effectively block or inactivate any step in branched chain amino acid (BCAA) degradation cycle. Non-limiting examples of such steps include transfer of the α-amino group to a-ketoglutarate to form glutamate through the activity of branched-chain amino acid aminotransferase (BCAT) or production of alanine by ALT. In some aspects, a chemical compound used herein can be any chemical compound that can block or reduce the activity of ALT. In some aspects, the chemical compound that can block or reduce the activity of ALT is β-chloro-L-alanine, one or more flavonoid (e.g., hesperetin, hesperidin), L-cycloserine, L-2-amino-4-methoxy-trans-but-3-enoic acid, aminooxyacetate (AOA) or epigallocatechin gallate (EGCG), a pharmaceutically acceptable salt, derivative or analog thereof, or any combination thereof.

In some aspects, an inhibitor of ALT comprises β-chloro-L-alanine, or a pharmaceutically acceptable salt, derivative or analog thereof. β-chloro-L-alanine (CAS No. 51887-89-9) can be synthesized using known methods in the art or can be obtained commercially (for e.g., Sigma Aldrich C9033). In some aspects, β-chloro-L-alanine, or a pharmaceutically acceptable salt, derivative or analog thereof, disrupts the tumor-liver alanine cycle. In some aspects, β-chloro-L-alanine, or a pharmaceutically acceptable salt, derivative or analog thereof, inhibits alanine production, impairs glucose uptake, or any combination thereof, in a cancer or tumor cell of the subject. In some aspects, β-chloro-L-alanine, or a pharmaceutically acceptable salt, derivative or analog thereof, inhibits alanine and/or glucose accumulation in a cancer or tumor cell, or a serum sample from the subject, by at least 20%, e.g., 30%, 40%, 50%, 60%, 70%, 80%, 90%, 95% or above, as compared to alanine and/or glucose accumulation before administration of the inhibitor (baseline) or a control cell/sample.

In some aspects, an inhibitor of ALT comprises an antibody or a biologically active fragment thereof that can inhibit ALT activity. Such antibodies can be made using any known method in the art. The wild type sequences of ALT are well known in the art and may be obtained from publicly available databases. For example, the amino acid sequence for human ALT is available at NCBI database accession number NP_001369594.1. In some aspects, antibody used herein can be an antibody derived from any non-human animals (such as mice, rats, rabbits, hamsters, dogs, sheep, and monkeys), human antibody, or the like. Recombinant antibodies, non-limiting examples of which include antibody artificially modified to reduce antigenicity in humans or for other purposes, such as chimeric antibody and humanized antibody can also be used. Antibodies as disclosed herein may also include modified antibodies obtained by conjugating antibodies with various molecules. The modified antibodies include antibodies conjugated with various molecules such as cytotoxic agents or polyethylene glycol (PEG). Non-limiting examples of cytotoxic agents include radioisotopes, chemotherapeutic agents, cellular toxins, etc. Such modified antibodies can be obtained by chemically modifying the antibodies produced as above. Methods for modifying antibodies have already been established in this field of art. In some aspects, antibody used herein may include small antibody fragments. Small antibody fragments include antibody fragments obtained by removing a part of whole antibodies (e.g., whole IgG, etc.) and are not specifically limited so far as they retain antigen-binding ability. In some aspects, antibody fragments may contain a heavy chain variable region (VH) or a light chain variable region (VL), or both VH and VL. Non-limiting examples of antibody fragments may include, e.g., Fab, Fab′, F(ab′)2, Fv, scFv (single-chain Fv), etc. Such antibody fragments can be obtained by treating antibodies with an enzyme such as papain or pepsin to produce antibody fragments or by constructing genes encoding these antibody fragments and introducing them into an expression vector and then expressing them in a suitable host cell. In some aspects, diabody is a dimer consisting of two fragments, each having variable regions joined together via a linker. In some aspects, the small antibody fragment comprises diabodies. In some aspects, diabody-forming fragments include those consisting of VL and VH, VL and VL, or VH and VH. In some aspects, diabody-forming fragments can be joined via a linker to form single-chain diabodies (sc(Fv)₂).

In some aspects, an inhibitor of ALT as disclosed herein comprises a biological inhibitor. In some aspects, a biological inhibitor is a molecularly, physically or chemically inactivated ALT protein that can compete with and/or reduce the activity of ALT. In some aspects, a biological inhibitor is an RNAi, including siRNA, miRNA, or dsRNA. Such inhibitors can be made using any known method in the art. The wild type sequences of ALT are well known in the art and may be obtained from publicly available databases. For example, the nucleic acid sequence for human ALT is available at NCBI database under accession number D10355.1.

In some aspects, the present disclosure encompasses pharmaceutically acceptable derivatives of the inhibitor of ALT including but not restricted to salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

In some aspects, the present disclosure encompasses pharmaceutically acceptable analogs of an inhibitor of ALT. The analog can be a small organic compound, a nucleotide, a protein, or a polypeptide that possesses similar or identical activity or function(s) as the compound, nucleotide, protein or polypeptide or compound having the desired activity and therapeutic effect (e.g., inhibition of tumor growth), but need not necessarily comprise a sequence or structure that is similar or identical to the sequence or structure of the parent or wild-type inhibitor.

### II. Compositions

In some aspects, the present disclosure encompasses a composition comprising an inhibitor of ALT. Suitable inhibitors are described above. In some aspects, a composition comprises an inhibitor of ALT, wherein the inhibitor is a chemical compound, antibody, or other biological inhibitor. In some aspects, the composition comprising an inhibitor of ALT may further comprise a pharmaceutical excipient.

In some aspects, a composition of the present disclosure comprises an inhibitor of ALT, wherein the inhibitor is a chemical compound. In some aspects, the composition comprises an inhibitor of ALT and a pharmaceutical excipient, wherein the inhibitor is a chemical compound. In some aspects, the composition comprises β-chloro-L-alanine, or a pharmaceutically acceptable salt, derivative or analog thereof. In some aspects, the composition comprises β-chloro-L-alanine, or a pharmaceutically acceptable salt, derivative or analog thereof, and a pharmaceutical excipient.

In some aspects, a composition may comprise from about 1 mg to about 50 g, from about 0.1 to about 5 g, from about 0.5 g to about 3 g, from about 1 mg to about 55 mg, from about 40 mg to about 60 mg, from about 80 mg to about 120 mg, from about 180 mg to about 220 mg, from about 0.1 g to about 5 g, or from about 0.5 g to about 3 g of β-chloro-L-alanine. In some aspects, a composition comprises β-Chloro-L-alanine or a pharmaceutically acceptable salt, derivative or analog thereof, at an amount of about 10 mg to about 2 grams.

In some aspects, a composition comprising an inhibitor of ALT can be formulated and administered to a subject by several different means. For instance, a composition can generally be administered parenteraly, intraperitoneally, intravascularly, transdermally, subcutaneously, or intrapulmonarily in dosage unit formulations containing conventional nontoxic pharmaceutically acceptable adjuvants, carriers, excipients, and vehicles as desired. The term parenteral as used herein includes subcutaneous, intravenous, intramuscular, intrathecal, or intrasternal injection, or infusion techniques. Formulation of pharmaceutical compositions is discussed in, for example, Hoover, John E., Remington’s Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

A pharmaceutical formulation comprises one or more pharmaceutically acceptable excipients. Non-limiting examples of excipients may include chemical enhancers, humectants, pressure sensitive adhesives, antioxidants, solubilizers, thickening agents, plasticizers, adjuvants, carriers, excipients, vehicles, coatings, and any combination thereof. One or more excipients can be selected for oral, transdermal, parenteral, intraperitoneal, intravascular, subcutaneous, by inhalation spray, rectal, or intrapulmonary administration. In some aspects, the pharmaceutically acceptable excipient comprises a binder, a filler, a disintegrant, a lubricant, a glidant, a salt, a polymer, buffering agent, solvent, or any combination thereof.

A composition described herein may conveniently be presented in unit dosage form and may be prepared by any methods well known in the art of pharmacy. The amount of active ingredient that can be combined with an excipient material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association an inhibitor compound as described herein with an excipient and, optionally, one or more accessory components. In general, the formulations are prepared by uniformly and intimately bringing into association an inhibitor compound described herein with liquid excipients, or finely divided solid excipients, or both, and then, if necessary, shaping the product.

Compositions described herein suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an inhibitor compound described herein as an active ingredient. A composition described herein may also be administered as a bolus, electuary, or paste.

In solid dosage forms described herein for oral administration (capsules, tablets, pills, dragees, powders, granules and the like), the active ingredient is mixed with one or more pharmaceutically acceptable excipients, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof, and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory components. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions described herein, such as dragees, capsules, pills and granules, may optionally be scored or prepared with coatings and shells, such as enteric coatings and other coatings well known in the pharmaceutical-formulating art. They may also be formulated to provide slow or controlled release of the active ingredient therein using, for example, hydroxypropylmethyl cellulose in varying proportions to provide the desired release profile, other polymer matrices, liposomes and/or micro-spheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds described herein include pharmaceutically acceptable emulsions, microemulsions, solutions, suspensions, syrups, or elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying agents, suspending agents, sweetening, flavoring, coloring, perfuming, preservative agents, or combinations thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound described herein include powders, sprays, ointments, pastes, creams, lotions, gels, solutions, patches and inhalants. The active compound may be mixed under sterile conditions with a pharmaceutically acceptable excipient, and with any preservatives, buffers, or propellants that may be required.

The ointments, pastes, creams, and gels may contain, in addition to an active inhibitor compound described herein, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an inhibitor compound described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and pro-pane.

Transdermal patches have the added advantage of providing controlled delivery of a compound described herein to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions described herein suitable for parenteral administration comprise one or more inhibitor compounds described herein in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous excipients that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, dispersing agents, or combinations thereof. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of an inhibitor compound, it is desirable to slow the absorption of the inhibitor compound from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally administered drug form is accomplished by dissolving or suspending the inhibitor compound in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The compositions described herein may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. In some aspects, the composition is administered orally, and/or IV administration. In some aspects, the composition is administered intraperitoneally.

In some aspects, the composition comprising an inhibitor of alanine aminotransferase is administered for treatment of cancer in a subject in need thereof. In some aspects, the composition comprises an inhibitor of alanine aminotransferase and at least a pharmaceutically acceptable excipient. In some aspects, the composition for treatment of cancer in a subject in need thereof comprises β-chloro-L-alanine, or a pharmaceutically acceptable salt, derivative or analog thereof. In some aspects, the composition for treatment of cancer in a subject in need thereof comprises β-chloro-L-alanine, or a pharmaceutically acceptable salt, derivative or analog thereof, and a pharmaceutical excipient.

In some aspects, the administration of a disclosed composition in the subject disrupts the tumor-liver alanine cycle. In some aspects, the administration of a disclosed composition in the subject effectively blocks or inactivates any step in the BCAA degradation cycle. In some aspects, the administration of a disclosed composition in the subject blocks or reduces the activity of ALT.

In some aspects, administration of a disclosed composition in the subject decreases ALT activity in a subject by at least 20%, e.g., 30%, 40%, 50%, 60%, 70%, 80%, 90%, 95% or above, as compared to baseline activity before administration of the inhibitor in the subject or a control.

In some aspects, the composition comprises an inhibitor of ALT, wherein the inhibitor is β-chloro-L-alanine, one or more flavonoid (e.g., hesperetin, hesperidin), L-cycloserine, L-2-amino-4-methoxy-trans-but-3-enoic acid, aminooxyacetate (AOA) or epigallocatechin gallate (EGCG), a pharmaceutically acceptable salt, derivative or analog thereof, or any combination thereof.

In some aspects, a composition of the present disclosure comprises β-chloro-L-alanine, or a pharmaceutically acceptable salt, derivative or analog thereof and administration of the composition to a subject disrupts the tumor-liver alanine cycle In some aspects, a composition comprises β-chloro-L-alanine, or a pharmaceutically acceptable salt, derivative or analog thereof and administration of the composition in the subject inhibits alanine production, impairs glucose uptake, or any combination thereof. In some aspects, composition comprises β-chloro-L-alanine, or a pharmaceutically acceptable salt, derivative or analog thereof and administration of the composition to a subject inhibits alanine and/or glucose accumulation in the subject, by at least 20%, e.g., 30%, 40%, 50%, 60%, 70%, 80%, 90%, 95% or above, as compared to alanine and/or glucose accumulation before administration of the inhibitor (baseline) or a control.

In some aspects, a composition of the present disclosure comprises β-chloro-L-alanine, or a pharmaceutically acceptable salt, derivative or analog thereof and administration of the composition to the subject reduces the dimensions of the tumor. In some aspects, administration of a composition of the present disclosure to a subject reduces one or more tumor dimensions by at least about 10% to about 50%. In some aspects, the dimensions of the tumor may comprise tumor size, tumor volume or tumor growth. In some aspects, a composition comprises β-chloro-L-alanine, or a pharmaceutically acceptable salt, derivative or analog thereof and administration of the composition to a subject in need thereof reduces tumor size, tumor volume or tumor growth. In some aspects, a composition of the present disclosure comprises β-chloro-L-alanine, or a pharmaceutically acceptable salt, derivative or analog thereof and administration of the composition to a subject reduces tumor size, tumor volume or tumor growth in the subject by at least 10%, e.g., 20%, 30%, 40%, 50%, 60%, 70%, 80%, 90%, 95% or above, as compared to tumor size, tumor volume or tumor growth before administration of the inhibitor (baseline) or a control. In some aspects, a composition of the present disclosure comprises β-chloro-L-alanine, or a pharmaceutically acceptable salt, derivative or analog thereof and administration of the composition to a subject reduces tumor size in the subject by at least 10%, e.g., 20%, 30%, 40%, 50%, 60%, 70%, 80%, 90%, 95% or above, as compared to tumor size before administration of the inhibitor (baseline) or a control.

In some aspects, a composition of the present disclosure is administered to a subject with melanoma, non-small cell lung carcinoma, nasopharyngeal carcinoma, ovarian cancer, breast cancer, hepatocellular carcinoma, chronic myeloid leukemia, or glioblastoma. In some aspects, a composition of the present disclosure is administered to a subject with melanoma.

### III. Method of Treatment

In further aspects, the disclosure provides a method for treatment of a tumor or a cancer in a subject. In some aspects, the method comprises administering to the subject in need thereof, an effective amount of a composition comprising an inhibitor of ALT. In some aspects, the method comprises administering to the subject in need thereof, an effective amount of a composition comprising an inhibitor of ALT, and at least a pharmaceutically acceptable excipient.

In some aspects, the method of treatment comprises administering a composition comprising any of the disclosed inhibitors of ALT detailed above. In some aspects, a method for treatment of a tumor or a cancer comprises administering to the subject in need thereof an effective amount of a composition comprising an inhibitor of ALT, wherein the inhibitor is a chemical compound, antibody, or other biological inhibitor. In some aspects, a method for treatment of a tumor or a cancer comprises administering to the subject in need thereof an effective amount of a composition comprising an inhibitor of ALT and a pharmaceutical excipient, wherein the inhibitor is a chemical compound, antibody, or other biological inhibitor.

In some aspects, the method for treatment of a tumor or a cancer comprises administering to the subject in need thereof an effective amount of a composition comprising an inhibitor of ALT, wherein the inhibitor is a chemical compound. In some aspects, the method for treatment of a tumor or a cancer comprises administering to the subject in need thereof an effective amount of a composition comprising an inhibitor of ALT, and a pharmaceutical excipient, wherein the inhibitor is a chemical compound. In some aspects of the method, the chemical compound is β-chloro-L-alanine, one or more flavonoids (e.g., hesperetin, hesperidin), L-cycloserine, L-2-amino-4-methoxy-trans-but-3-enoic acid, aminooxyacetate (AOA) or epigallocatechin gallate (EGCG), a pharmaceutically acceptable salt, derivative or analog thereof, or any combination thereof. In some aspects, a method for treatment of a tumor or a cancer comprises administering to the subject in need thereof an effective amount of a composition comprising β-chloro-L-alanine, or a pharmaceutically acceptable salt, derivative or analog thereof. In some aspects, a method for treatment of a tumor or a cancer comprises administering to the subject in need thereof, an effective amount of a composition comprising β-chloro-L-alanine, or a pharmaceutically acceptable salt, derivative or analog thereof, and a pharmaceutical excipient.

In some aspects, a method for treatment of a tumor or a cancer comprises administering to the subject in need thereof, a composition which comprises from about 1 mg to about 50 g, from about 0.1 g to about 5 g, from about 0.5 g to about 3 g, from about 1 mg to about 55 mg, from about 40 mg to about 60 mg, from about 80 mg to about 120 mg, from about 180 mg to about 220 mg, from about 0.1 g to about 5 g, or from about 0.5 g to about 3 g of β-chloro-L-alanine. In some aspects, a method for treatment of a tumor or a cancer comprises administering to the subject in need thereof a composition which comprises β-Chloro-L-alanine or a pharmaceutically acceptable salt, derivative or analog thereof, at an amount of about 10 mg to about 2 grams. In some aspects of the method, the β-chloro-L-alanine is administered at a unit dose amount selected from a range of 10 mg to about 2 grams.

In some aspects, the effective amount of inhibitor of ALT can range from about 0.5 mg to about 20 mg, about 1 mg to about 60 mg, about 30 mg to about 50 mg, or about 3 mg to about 5 mg. In some aspects, the effective amount of inhibitor of ALT can range from about 0.5 mg/day to about 100 mg/day, from about 1 to about 60 mg/day, from about 20 to about 50 mg/day, from about 20 to about 30 mg/day, or from about 15 to about 25 mg/day. In some aspects, the effective amount of β-chloro-L-alanine, is from about 0.5 mg/day to about 100 mg/day, from about 1 to about 60 mg/day, from about 20 to about 50 mg/day, from about 20 to about 30 mg/day, or from about 15 to about 25 mg/day.

Compositions comprising inhibitors of ALT disclosed herein can be administered to the subject daily or more than once daily. In some aspects, β-chloro-L-alanine can be administered every 2, 3, 4, 5, 6, 7, 14, or every 30 days. β-chloro-L-alanine can be administered over a period ranging from about 1 day to about 1 year, from about 1 day to about 1 week, from about 3 days to about 1 month, from about 2 weeks to about 6 months, or from about 2 months to about 4 months. β-chloro-L-alanine can also be administered over a period of about 1 day, about 7 days, about 30 days, about 60 days, about 120 days, or about 180 days or more. In some aspects, β-chloro-L-alanine is administered over a period of about 57 weeks, about 148 weeks, about 208 weeks, or indefinitely.

Actual dosage levels of the inhibitors described herein may be varied to obtain an amount that is effective to achieve the desired therapeutic response for a particular subject, composition, and mode of administration, without being toxic to the subject. A physician or veterinarian having ordinary skill in the art can readily determine and prescribe the effective amount of the inhibitor required. For example, the physician or veterinarian could start doses of the compounds described herein employed in the pharmaceutical composition at levels lower than that required in order to achieve the desired therapeutic effect and gradually increase the dosage until the desired effect is achieved.

In some aspects, a composition comprising an inhibitor of ALT or a pharmaceutically acceptable salt, derivative or analog thereof, can be administered by a mode selected from parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, and transdermal.

In some aspects, β-chloro-L-alanine or a pharmaceutically acceptable salt, derivative or analog thereof is administered by a mode selected from parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, and transdermal.

In some aspects, a method of treatment by administration of the composition comprising the ALT inhibitor, or a pharmaceutically acceptable salt, derivative or analog thereof, to the subject, disrupts the tumor-liver alanine cycle. In some aspects, a method of treatment by administration of a composition comprising β-chloro-L-alanine, or a pharmaceutically acceptable salt, derivative or analog thereof, to the subject inhibits alanine production, impairs glucose uptake, or any combination thereof. In some aspects, a method of treatment by administration of a composition comprising β-chloro-L-alanine, or a pharmaceutically acceptable salt, derivative or analog thereof, to the subject inhibits alanine and/or glucose accumulation in the subject, by at least 20%, e.g., 30%, 40%, 50%, 60%, 70%, 80%, 90%, 95% or above, as compared to alanine and/or glucose accumulation before administration of the inhibitor (baseline) or a control.

In some aspects, a method of treatment by administration of a composition comprising an inhibitor of ALT, or a pharmaceutically acceptable salt, derivative or analog thereof, to the subject reduces at least one dimension of the tumor. In some aspects, administration of a composition to the subject reduces one or more tumor dimensions by at least about 10% to about 50%. In some aspects, the one or more dimensions of the tumor may comprise tumor size, tumor volume or tumor growth. In some aspects, a method of treatment by administration of a composition comprising β-chloro-L-alanine, or a pharmaceutically acceptable salt, derivative or analog thereof, to the subject reduces one or more of the tumor size, tumor volume or tumor growth. In some aspects, a method of treatment by administration of a composition comprising β-chloro-L-alanine, or a pharmaceutically acceptable salt, derivative or analog thereof, to the subject reduces one or more of tumor size, tumor volume or tumor growth in the subject by at least 10%, e.g., 20%, 30%, 40%, 50%, 60%, 70%, 80%, 90%, 95% or above, as compared to tumor size, tumor volume or tumor growth before administration of the inhibitor (baseline) or a control. In some aspects, a method of treatment by administration of a composition comprising β-chloro-L-alanine, or a pharmaceutically acceptable salt, derivative or analog thereof, to the subject reduces tumor size in the subject, by at least 10%, e.g., 20%, 30%, 40%, 50%, 60%, 70%, 80%, 90%, 95% or above, as compared to tumor size before administration of the inhibitor (baseline) or a control.

In some aspects, a method of treatment disclosed herein can effectively reduce one or more symptoms associated with cancer. In some aspects, the symptoms are selected from the group consisting of abnormal bodily function, abnormal cell morphology, abnormal enzyme levels, abnormal hormone levels, abnormal oncofetal antigen levels, abnormal tissue growth, abnormal tissue mass, abnormal tumor-associated protein levels, altered neurologic function, altered neurologic structure, angiogenesis, bleeding, cells having a cancer cell phenotype, diarrhea, effusions, fatigue, fever, lesions, malnutrition, metastasis, nausea, obstruction of a bodily passageway, opportunistic infection, pain, poor Karnofsky performance status, presence of cell-surface markers, presence of histological markers, presence of intracellular markers, presence of molecular markers, tumor invasion, unregulated cell proliferation, urinary frequency, and weight loss. In some aspects of the method, one or more symptoms is reduced by at least 10%, e.g., 20%, 30%, 40%, 50%, 60%, 70%, 80%, 90%, 95% or above, as compared to the symptom(s) before administration of the inhibitor (baseline) or a control.

In some aspects, a method for treatment of a tumor or a cancer comprises administering to the subject an effective amount of a composition comprising an inhibitor of ALT to the subject, wherein the subject has melanoma, non-small cell lung carcinoma, nasopharyngeal carcinoma, ovarian cancer, breast cancer, hepatocellular carcinoma, chronic myeloid leukemia, or glioblastoma. In some aspects, a method for treatment comprises administering a composition comprising a disclosed inhibitor of ALT to a subject with melanoma. In some aspects, a method comprises administering an effective amount of a composition comprising β-chloro-L-alanine, or a pharmaceutically acceptable salt, derivative or analog thereof, to a subject with melanoma.

In some aspects, a method of treatment further comprises administering an additional cancer therapy to the subject. The additional cancer therapy can comprise immunotherapy, surgery, chemotherapy and/or radiotherapy. Such therapies can be administered concurrently or non-concurrently with administration of a composition comprising a disclosed inhibitors of ALT.

In some aspects, a disclosed method can comprise modifying one or more of the administrations of a composition comprising an inhibitor of ALT. For example, one or more features or aspects of one or more steps of a method disclosed herein may be modified or changed. For example, in an aspect, a method may be altered by changing the amount administered of a composition comprising one or more of adisclosed inhibitor of ALT, by changing the pharmaceutical formulation, or by a combination thereof, or by changing the frequency of administration of a composition comprising one or more of a disclosed inhibitor of ALT, pharmaceutical formulations, or a combination thereof, or by changing the duration of administration time for a composition comprising one or more of a disclosed inhibitor of ALT.

### IV. Kits

In some aspects, a composition comprising an inhibitor of ALT described herein can be provided in a kit. The kit includes a composition described herein, e.g., a composition that includes β-chloro-L-alanine, or a pharmaceutically acceptable salt, derivative or analog thereof, and informational material. The informational material can be descriptive, instructional, marketing or other material that relates to the methods described herein and/or the use of a composition comprising an inhibitor of ALT, for the methods described herein. For example, the informational material may describe methods for administering the inhibitor of ALT, to treat cancer, or reduce at least one symptom of the cancer.

In some aspects, the informational material can include instructions to administer a composition of the present disclosure in a suitable manner, e.g., in a suitable dose, dosage form, or mode of administration (e.g., a dose, dosage form, or mode of administration described herein). The informational material of the kits is not limited in its form. In many cases, the informational material, e.g., instructions, is provided in printed matter, e.g., a printed text, drawing, and/or photograph, e.g., a label or printed sheet. However, the informational material may also be provided in other formats, such as Braille, computer readable material, video recording, or audio recording. In some aspects, the informational material of the kit is a link or contact information, e.g., a physical address, email address, hyperlink, website, qr code or telephone number, where a user of the kit can obtain substantive information about the composition comprising an inhibitor of ALT, and/or its use in the methods described herein.

In addition to an inhibitor of ALT, a composition of the kit can include other components, such as a solvent or buffer, a stabilizer or a preservative, and/or a second agent for treating cancer. Alternatively, the other components may be included in the kit, but in different compositions or containers than the inhibitor of ALT. In such aspects, the kit can include instructions for admixing the composition comprising an inhibitor of ALT, and the other components, or for using the composition comprising an inhibitor of ALT, together with the other components.

A composition comprising an inhibitor of ALT may be provided in any form, e.g., liquid, dried or lyophilized form. In some aspects, a composition comprising an inhibitor of ALT is substantially pure and/or sterile. When a composition comprising an inhibitor of ALT is provided in a liquid solution, the liquid solution preferably is an aqueous solution, with a sterile aqueous solution. When a composition comprising an inhibitor of ALT is provided as a solid form, reconstitution generally is by the addition of a suitable solvent. In some aspects, the solvent, e.g., sterile water or buffer (e.g., PBS), may be provided in the kit.

The kit can include one or more containers for the composition comprising the inhibitor of ALT. In some aspects, the kit comprises separate containers, dividers or compartments for the composition comprising an inhibitor of ALT and informational material. For example, a composition comprising an inhibitor of ALT can be contained in a bottle, vial, or syringe, and the informational material can be contained in a plastic sleeve or packet. In other aspects, the separate components of the kit are contained within a single, undivided container. For example, a composition comprising an inhibitor of ALT is contained in a bottle, vial or syringe that has attached thereto the informational material in the form of a label. In some aspects, the kit includes a plurality (e.g., a pack) of individual containers, each containing one or more unit dosage forms (e.g., a dosage form described herein) of the agent (e.g., in a composition). For example, the kit can include a plurality of syringes, ampules, or foil packets, each containing a single unit dose of a composition comprising an inhibitor of ALT. The containers of the kits may be airtight and/or waterproof.

Having described several aspects, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

### V. Laboratory Animal Tumor Model

Some aspects of the disclosure provides a laboratory animal tumor model. In some aspects, the disclosure encompasses a method of analysis of tumor metabolism in a laboratory animal tumor model. In some aspects, the method comprises continuous administration of an isotope tracer over a period of time through direct ingestion of fluids, obtaining a blood sample from the laboratory animal, conducting a metabolomics analysis using LC/MS, and comparing the hepatic gluconeogenesis flux in the laboratory animal tumor model with the hepatic gluconeogenesis flux of a healthy animal model without tumor.

In some aspects, the laboratory animal is a zebra fish. Suitable zebrafish strains include the wild-type strains such as AB, Tubingen, AB/TUbingen, Sanger AB Tubingen, SJD, SJA, WIK strains, and the pigmentation mutant strains such as golden, albino, rose, panther, leopard, jaguar, puma, bonaparte, cezanne, chagall, dali, duchamp, picasso, seurat, sparse, shady, oberon, opallus, nacre, roy, and Casper strains.

In some aspects, the zebra fish is a wild-type strain (e.g., inbred AB strain). In some aspects, the zebra fish is a genetically modified fish or a transgenic fish. In some aspects, the zebra fish is a transgenic fish genetically modified to express a nucleic acid encoding a mutant proto-oncogene and/or a nucleic acid encoding a mutant human BRAF protein.

In further aspects, the zebra fish is allowed to swim freely in facility water containing the stable isotopes, non-limiting example of which is ¹³C glucose. The fish may then be used to evaluate candidate compounds or agents that affect hepatic gluconeogenesis, and associated cancer treatment effectiveness. For example, the candidate compound can be introduced into the water and zebra fish can placed in the tank of treated water. After being under suitable conditions for a suitable period, zebra fish may be evaluated for alteration in gluconeogenesis, by a method including but not limited to procuring blood or serum from the zebra fish, followed by metabolite extraction from the blood or serum, and analysis of the metabolites involved in gluconeogenesis using LC/MS, and which can be compared with a control zebra fish that were not exposed to the candidate compound and/or healthy zebra fish. In further aspects, zebra fish may be evaluated for tumor size, growth and/or volume, compared with a control zebra fish that were not exposed to the candidate compound, and/or healthy zebra fish. If the metabolites or development of tumor in the presence of the candidate molecule is altered as compared to that in the absence of the candidate molecule, it indicates that the candidate molecule may affect hepatic gluconeogenesis, and growth and development of cancer.

Those skilled in the art will appreciate that the presently disclosed aspects teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate various aspects of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Methods and Materials

Liquid chromatography/mass spectrometry (LC/MS)-grade, Burdick & Jackson brand water, acetonitrile, and methanol were purchased from Honeywell (Muskegon, MI). LC/MS-grade eluent ammonium acetate, LC/MS-grade ammonium hydroxide solution (25%), methylenediphosphonic (medronic) acid, and tricaine methanesulfonate were purchased from Sigma-Aldrich (St. Louis, MO). TraceSELECT Fluka brand ammonium monobasic was purchased from Honeywell (Muskegon, MI). Chemical standards, including 2-aminoisobutyric acid, 2-deoxyglucose, and 2-deoxyglucose 6-phosphate, were purchased from Sigma-Aldrich (St. Louis, MO).

### Zebrafish Husbandry

This study was carried out in accordance with the Washington University Institutional Animal Care and Use Committee (IACUC) regulations. Wild-type (WT) fish were inbred AB strain (sjA). p53-/- and transgenic mitfa-BRAFV600E¬ lines were crossed to produce melanoma-bearing fish (referred to as BRAF/p53), using a previously described method. All fish were reared according to standard laboratory procedures. Fish were kept in an indoor environment at a temperature of 28 ± 1° C. with a 14:10-h light:dark circadian cycle. Both female and male fish were used for experiments at random. Experiments were conducted with age-matched animals (i.e., within 2 weeks of age). To assay tumor growth, melanoma dimensions were measured by using digital calipers. Tumor size was approximated as the volume of a half ellipsoid, ⅔ πabc, where a, b, and c represent the radii of tumor length, width, and height respectively.

To anesthetize by gradual cooling, zebrafish were initially placed in a beaker containing 100 mL of water at 17° C. The beaker containing the fish was then placed in a shallow ice bath to allow for gradual cooling over the course of 5 min down to 12° C. until stage III, phase 2 anesthesia was achieved, characterized by loss of reactivity, loss of balance, and loss of operculum movements. For experiments testing tricaine anesthesia, fish were immersed in 0.02% MS-222 in facility water for 1 - 2 min until stage III, phase 2 anesthesia was achieved. Euthanasia was performed by placing fish in an ice-water slurry for at least 10 min and was deemed complete 1 min after the cessation of opercular movements.

### Serum Harvest

For blood collection method development, we evaluated two protocols. First, blood was pipetted directly from a zebrafish following wound generation as previously described. Briefly, an anesthetized zebrafish was removed from water and dried thoroughly with a Kimwipe. A razor blade was used to amputate the caudal fin and some associated distal tissue by making a transverse cross section midway between the anal and caudal fin. Upon caudal fin amputation, blood was pipetted directly from the wound, with particular focus on the dorsal aorta region, using tips of various sizes.

To evaluate a second protocol that employed low-speed centrifugation, a simple blood-collection device was prepared by forming a small hole in the bottom of a 1.5 mL microcentrifuge tube (the holding tube) with a razor blade. The hole was approximately the diameter of the cross section created via caudal fin amputation (~0.5 mm). The holding tube was fit into a 0.5 mL microcentrifuge tube (the collection tube) and secured with adhesive. The holding tube attached to the collection tube constituted the blood-collection apparatus. To collect blood, zebrafish were anesthetized and dried thoroughly. A razor blade was used to create a wound, as described above. Upon caudal fin amputation, zebrafish were immediately placed (wound-side down) into the holding tube of the blood-collection apparatus. The collection apparatus containing a wounded fish was placed into a microcentrifuge and spun at 40 g and 15° C. for 30 s to collect blood. This method was adapted from a previously described method to accommodate the time-sensitive nature of metabolic experiments.

Immediately following all blood collections, blood was allowed to clot on ice for 10 min and then centrifugated at 1,600 g and 4° C. for 10 min to separate serum from blood cells. Serum was transferred to a new 0.5 mL microcentrifuge tube, snap frozen in liquid nitrogen, and stored at -80° C.

### Isotope Tracing

For all isotope tracing experiments, zebrafish were fasted for 24 h to empty the digestive tract prior to labeling. To determine the [U-¹³C] glucose concentration for isotopic steady-state labeling, concentrations between 5 - 20 mM were tested. It was determined that 10 mM [U-¹³C] glucose was optimal, as it resulted in ~10% serum enrichment at isotopic steady state without perturbing circulating glucose levels. Similarly, concentrations between 1 - 10 mM of [U-¹³C] glutamine were tested for steady-state labeling. For glutamine labeling experiments, 5 mM [U-¹³C] glutamine was deemed optimal, as it yielded ~10-15% enrichment in serum glutamine without significantly altering circulating levels of glutamine. For ¹⁵N-BCAA labeling experiments, 2 mM each [¹⁵N] leucine, [¹⁵N] isoleucine, and [¹⁵N] valine were concurrently supplemented to facility water. For ¹³C-BCAA labeling experiments, 4 mM each of [U-¹³C] leucine, [U-¹³C] isoleucine, and [U-¹³C] valine were concurrently supplemented to facility water. During steady-state labeling experiments, zebrafish were allowed to swim freely in facility water containing the corresponding stable isotopes at the aforementioned concentrations, in addition to 2% penicillin/streptomycin (Life Technologies). For isotope tracing via intraperitoneal injection, zebrafish were injected using a previously described method. Briefly, anesthetized fish were quickly dried and weighed. Zebrafish were then placed into the trough of a wet precut sponge, with the abdomen facing up and gills secured in the trough. A 35G beveled needle and NanoFil 10 µL syringe (World Precision Instruments) was carefully inserted midline near the pelvic girdle, and a [U-¹³C] glucose treatment solution (0.5 mg/g) was slowly dispensed. After injection, fish were placed in 28° C. facility water and monitored for recovery from anesthesia (typically 5-10 seconds). Throughout the experiment, each fish was kept in an individual tank to allow for tracking.

All glassware that was used to carry out labeling experiments was washed thoroughly and autoclaved prior to experimentation. For the duration of the 24 h of labeling, fish were kept in an indoor environment at a temperature of 28 ± 1° C. with the same 14:10-h light:dark circadian cycle as described above. After 24 h of labeling, fish were anesthetized by gradual cooling, and tissues were harvested in pre-weighed 1.5 mL microcentrifuge tubes, snap frozen in liquid nitrogen, and stored at -80° C.

### Metabolite Extraction

To extract metabolites from serum, samples were diluted 1:15 with methanol:acetonitrile:water (2:2:1) at -20° C., vortexed for 10 s, and incubated at -20° C. for 1 h. Following incubation, metabolite extracts were centrifuged at 20,000 g and 4° C. for 10 min, and the supernatant was transferred into an LC/MS vial for same-day analysis. To extract metabolites from zebrafish tissues, frozen samples were ground in 1.5 mL microcentrifuge tubes with a disposable pellet pestle (Fisherbrand). The pellet pestle was placed in liquid nitrogen prior to sample grinding, and the microcentrifuge tube (containing tissue) was submerged in liquid nitrogen for the entirety of tissue grinding. We note that microcentrifuge tubes were labeled and weighed prior to tissue harvest and then weighed again following tissue grinding. The powderized tissue was mixed with -20° C. methanol:acetonitrile:water (2:2:1) and subjected to two cycles of freezing in liquid nitrogen (1 min), thawing in 25° C. water (10 s), sonication (5 min), and vortexing (30 s). Samples were then incubated at -20° C. for 1 h. For every 1 mg of tissue wet weight, 40 µL of extraction solvent was added. Following protein precipitation, tissue extracts were centrifuged at 20,000 g and 4° C. for 10 min, and the supernatant was transferred into an LC/MS vial for same-day analysis.

### LC/MS Analysis

Ultra-high performance LC (UHPLC)/MS was performed with a Thermo Scientific Vanquish Horizon UHPLC system interfaced with a Thermo Scientific Orbitrap ID-X Tribrid Mass Spectrometer (Waltham, MA). Hydrophilic interaction liquid chromatography (HILIC) was conducted with a SeQuant ZIC-pHILIC guard column (20 mm × 2.1 mm, 5 µm) connected to a SeQuant ZIC-pHILIC column (100 mm × 2.1 mm, 5 µm; EMD Millipore, Burlington, MA). Chromatographic solvents were adapted from a previously described work. Briefly, mobile phase solvents were composed of A = 20 mM ammonium acetate and 0.1% ammonium hydroxide in water:acetonitrile (95:5) and B = acetonitrile:water (95:5). Either 5 µM ammonium phosphate or 4 µM medronic acid was included in the A solvent. The following linear gradient at a flow rate of 0.25 mL/min: 0 -1 min, 90% B; 1 - 17.5 min, 45% B; 17.5 - 19 min, 30% B; 19 - 19.5 min, 30% B; 19.5 - 22 min, 90% B were applied. The column was equilibrated with 20 column volumes of 90% B following each injection. The column compartment was maintained at 40° C. and injection volumes were 4 µL for all experiments. Data were collected with the following MS source settings: spray voltage, -3 kV; sheath gas, 35; auxiliary gas, 10; sweep gas, 1; ion transfer tube temperature, 275° C.; vaporizer temperature, 300° C.; mass range, 67 - 1000 Da; resolution, 120,000 (MS1), 60,000 (MS/MS); maximum injection time, 100 ms; isolation window, 1.5 Da. With this method, it was possible to resolve ¹⁵N and ¹³C peaks for metabolites of interest. Metabolites were identified on the basis of accurate mass, MS/MS data, and retention time. All MS/MS data and retention times from compounds in research samples were matched to MS/MS data and retention times of purchased standards.

### Histology

Adult zebrafish were anesthetized via gradual cooling, and livers were rapidly dissected and placed on tin foil on dry ice. Fresh-frozen livers were embedded in carboxymethylcellulose (Sigma Aldrich) in water (5%), solidified at -80° C. for 1 h, and then stored at -20° C. overnight. Embedded tissues were sectioned at 5 µM thickness, mounted onto glass slides, and dehydrated until staining. Glycogen staining was performed by using a Periodic acid-Schiff (PAS) Kit (Sigma Aldrich) with and without diastase according to the manufacturer’s instructions. Samples were viewed on a Zeiss AxioObserver D1 inverted microscope (Carl Zeiss Inc. Thrownwood, NY) equipped with an Axiocam 503 color camera. Appropriate areas of the tissue were identified, and images were acquired with Plan-Apochromat (NA 1.4) oil objective by using the ZEN 2 (blue edition) software

### Real-Time PCR Analysis

RNA was extracted from zebrafish liver samples by using Trizol (Invitrogen). cDNA was synthesized with the SuperScript III First-Strand Synthesis SuperMix (Invitrogen) following the manufacturer’s guidelines. Reverse transcripts were produced by using SuperScript II (Invitrogen), and real-time reverse-transcription polymerase chain reaction was performed by using the PowerUp SYBR Green Master Mix (Bio-Rad) with a StepOnePlus Real-Time PCR system (Applied Biosystems) according to the manufacturer’s guidelines. Both elongation factor 1 alpha (EF1α) and β-actin were used as reference genes, and all primer sequences used are listed in the Table 1. All samples were run in triplicate and results were analyzed by using the 2^{-DDCt} method.

TABLE 1

Primers used for Real-time PCR analysis

| Gene | Primers (5′ to 3′) | GenBank No. |
|---|---|---|
| Elongation factor 1 α | F: CCCCTGGACACAGAGACTTCATC (SEQ ID NO:1) | L23807.1 |
| | R: ATACCAGCCTCAAACTCACCGAC (SEQ ID NO:2) | |
| β-actin | F: TCTGGTGATGGTGTGACCCA (SEQ ID NO:3) | AY222742 |
| | R: GGTGAAGCTGTAGCCACGCT (SEQ ID NO:4) | |
| Glucose 6-phosphatase | F: TGGCAGTGATAGGAGATTGGCTT (SEQ ID NO:5) | BC148168.1 |
| | R: AGTAGGACGTCTCATGGACCCAC (SEQ ID NO:6) | |
| Phosphoenolpyruvate carboxykinase 1 | F: ATCGCATCACGCATCGCTAAA (SEQ ID NO:7) | NM_214751 |
| | R: CCGCTGCGAAATACTTCTTCTGT (SEQ ID NO:8) | |

### Ammonia Excretion Assay

Groups of WT and tumor-bearing fish were individually housed in separate tanks of fresh facility water for 24 h, without circulation. Water samples were collected at baseline and after 24 h. The samples were frozen at -80° C. until analysis. The concentration of ammonia in water was determined by using a modified Berthelot assay. Briefly, 20 µL of sampled water was mixed with 400 µL of 100 mM phenol, 50 mg/L sodium nitroprusside, 125 mM sodium hydroxide, 0.38 M sodium phosphate dibasic, and 1% sodium hypochlorite. Following mixing, samples were incubated at 37° C. for 40 min, and then absorbance was read at 670 nm. An ammonium chloride standard curve was used to quantify ammonia.

### Glycogen Assay

WT and melanoma-bearing zebrafish were fasted for 24 h and then anesthetized, as described above, to harvest livers. Tissues were homogenized by using a disposable pellet pestle (Fisherbrand), suspended in 200 µL water, and boiled for 10 min to deactivate enzymes before analysis. Glycogen was quantified by using a commercial kit (Abcam ab65620) following the manufacturer’s protocol. Liver protein content was determined by using a Bradford Assay with Bradford Reagent (Bio-Rad), according to the manufacturer’s guidelines and a BSA standard curve.

### Analysis of Liver Weight

WT and melanoma-bearing zebrafish were anesthetized and weighed, and then livers were harvested in pre-weighed microcentrifuge tubes, as described above. Liver masses were determined by subtracting the combined tissue-microcentrifuge tube mass from the pre-weighed microcentrifuge tube mass. A ratio of liver to whole-body masses of WT and melanoma-bearing zebrafish were then compared. To account for the contribution of tumors to whole-body mass, the ratio of liver to whole-body mass after subtracting the mass of tumors was also calculated. Tumor masses were determined by using an analytical balance, after isolated tumors were transferred to pre-weighed microcentrifuge tubes.

### Vemurafenib Treatment

Adult zebrafish were treated with 100 mg/kg vemurafenib (Selleckchem) or DMSO (Sigma-Aldrich) once per day for 4 or 10 consecutive days. Drug dosage was selected based on amounts, which showed efficacy and no toxicity in zebrafish. Vemurafenib was dissolved in DMSO, and vemurafenib solution or DMSO alone (vehicle) was delivered via intraperitoneal injection following a previously described protocol detailed above. Throughout the experiment, each fish was kept in an individual tank to allow for tracking. After the final treatment, fish were fasted for 24 h and then exposed to 10 mM [U-¹³C] glucose for 24 h. Serum and tissues were harvested, immediately snap frozen in liquid nitrogen, and stored at -80° C. until LC/MS analysis. For ammonia quantification experiments, vehicle or vemurafenib solution was delivered to tumor-bearing zebrafish, as described above, for 4 or 10 consecutive days prior to water sampling.

### EIPA Treatment

Adult zebrafish were treated with 50 µM 5-(N-ethyl-N-isopropyl)amiloride (EIPA, Sigma-Aldrich) or DMSO (Sigma Aldrich). Drug dosage was adapted from previous work to avoid toxicity in tumor-bearing animals, and administered in the same manner as described in previous studies. Briefly, EIPA was dissolved in DMSO, and the drug solution was administered such that tank water was 0.1% drug solution v/v. For vehicle, DMSO alone was used. Tumor-bearing fish were randomly placed in either EIPA or vehicle conditions for a total of 6 days, with water changes every other day. Fish were fasted for 24 h beginning on the fourth day of treatment, and then exposed to 10 mM [U-¹³C] glucose for 24 h. Serum and tissues were harvested, immediately snap frozen in liquid nitrogen, and stored at -80° C. until LC/MS analysis.

### β-Chloroalanine Treatment

To determine the maximum nonlethal dose of β-chloroalanine, BRAF/p53 fish were housed in β-chloroalanine mixed with facility water at concentrations ranging from 0.05 - 2.0 mM. Animals were housed in β-chloroalanine-treated water for up to 5 days, with water changes every other day. Tanks were monitored frequently for animals that were experiencing acute toxicity or animals that had succumb to drug exposure, which were removed. A normal feeding schedule was maintained for all fish throughout the duration of drug treatment.

For ¹³C-BCAA labeling experiments following drug treatment, adult zebrafish were treated with 600 µM β-chloroalanine (Sigma-Aldrich) or vehicle. β-chloroalanine was dissolved directly in facility water, and tumor-bearing fish were randomly placed in either β-chloroalanine or no-drug (vehicle) conditions for a total of 10 days, with water changes every other day. Fish were fasted for 24 h beginning on the ninth day of treatment, and then exposed to 4 mM each of [U-¹³C] leucine, [U-¹³C] isoleucine, and [U-¹³C] valine for 24 h. Serum and tissues were harvested, immediately snap frozen in liquid nitrogen, and stored at -80° C. until LC/MS analysis.

### 2-Deoxyglucose and 2-Aminoisobutyric Acid Assay

Tumor-bearing fish were placed in facility water containing 0.5 mM 2-aminoisobutyric acid (2-AIB) or 1 mM 2-deoxyglucose (2-DG) for 8 h. Fish were anesthetized, and tissues were harvested and frozen as described above. Metabolites were extracted by using the above protocol and analyzed by LC/MS. Chemical standards of 2-aminoisobutyric acid and 2-deoxyglucose 6-phosphate were used to confirm metabolite identities.

### Analysis of BCAT1 Expression in Zebrafish

Differential expression of the BCAT1 gene was compared between melanoma cells and melanocytes by using RNA-seq. RNA was isolated from melanoma tumors from BRAF/p53 zebrafish and sorted melanocytes from WT zebrafish by using the Macherey Nagel Nucleospin XS kit (Fischer Scientific cat#NC0389511). The Genome Technology Access Center (GTAC) prepared the samples with a Clontech SMARTer cDNA amplification kit and ran sequencing by using an Illumina HiSeq 3000 system with 1×50bp read length. GTAC performed preliminary computational data analysis by aligning reads to zv9.

Resected surgical melanoma tumor samples were collected in RPMI 1640 media (Gibco, USA) and placed on ice. Tissue was dissociated into multiple pieces, and three to four ~1 cm pieces were flash frozen in tinfoil with liquid nitrogen. At a later date, specimens were removed from the freezer for processing. Total RNA was extracted by using RNAeasy Mini kits (Qiagen) following the manufacturer’s protocol. RNA samples were treated with RNase-free DNase (RQ1, Promega) and stored at -80° C. A Tru-Seq RNA library was constructed and NovaSeq S4 was used for sequencing, with a target of 50 million read pairs per library.

### Analysis of BCAT1 Expression in Human Patients

Samples were quantified by using Kallisto v0.43.1 with human genome reference GRCh38 (GRCh38-2.1.0). For gene-level expression estimates, the sum of the TPM values were taken for transcripts of the same gene. Human melanocytes RNA-seq data were obtained from previous studies. For comparison, FPKM was converted to TPM by TPM = (FPKM / sum of FPKM over all genes) * 10^6. RPKM was also converted to TPM with the same formula.

### Data Analysis

LC/MS data were processed and analyzed with the open-source Skyline program as well as Compound Discoverer 3.0 software (Thermo Scientific). Natural-abundance correction of ¹³C for tracer experiments was performed with AccuCor. The number of biological replicates in each experiment are listed in figure legends and were chosen based on previous experience. Statistical analyses of LC/MS data are described in figure legends.

### Example 1: Isotope Tracing in Single Adult Zebrafish

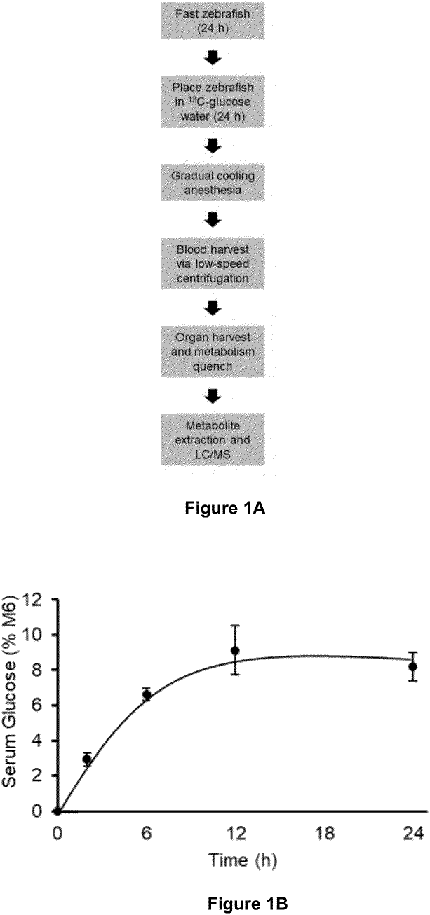

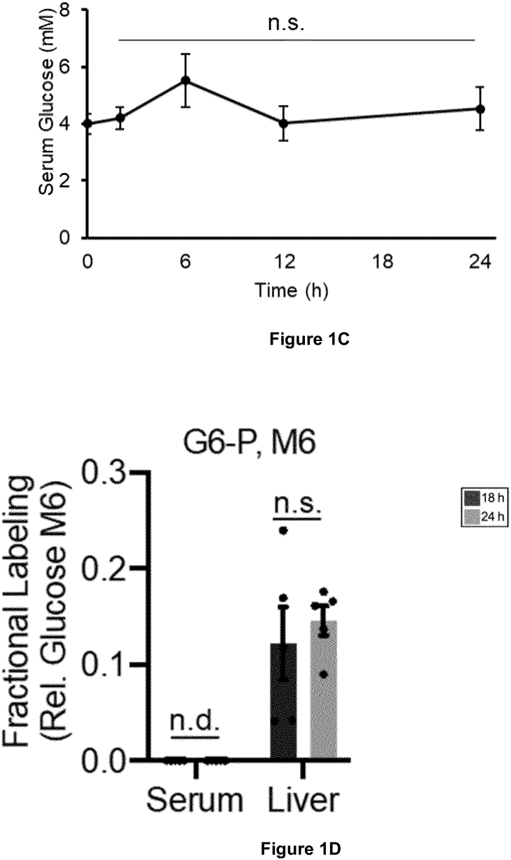

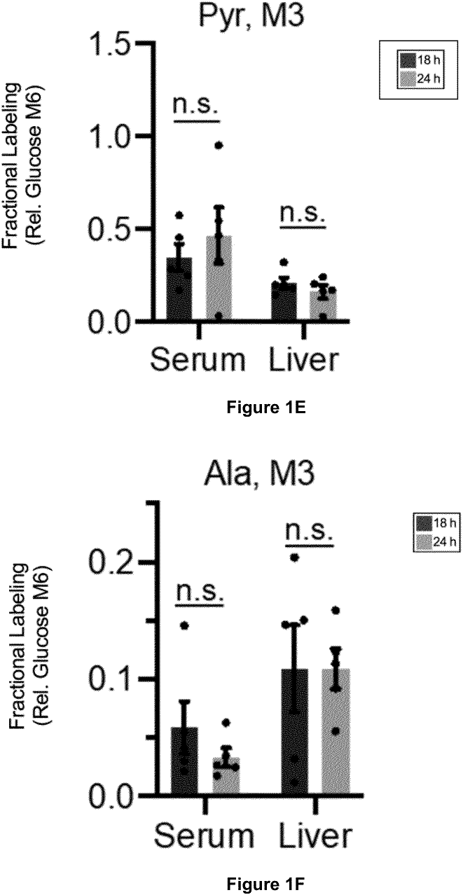

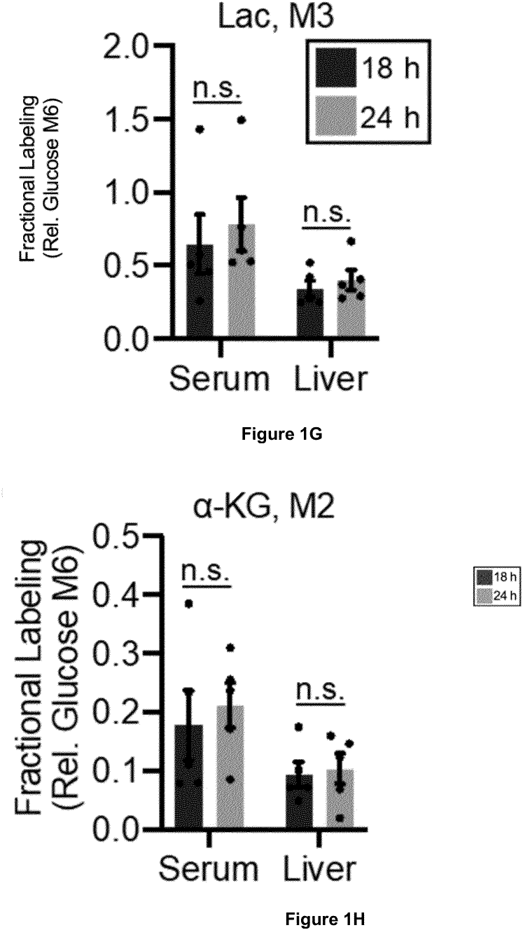

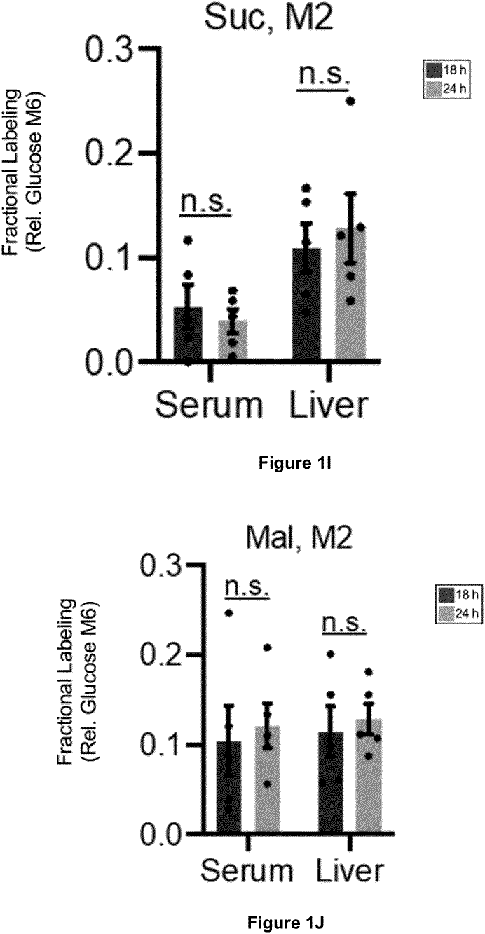

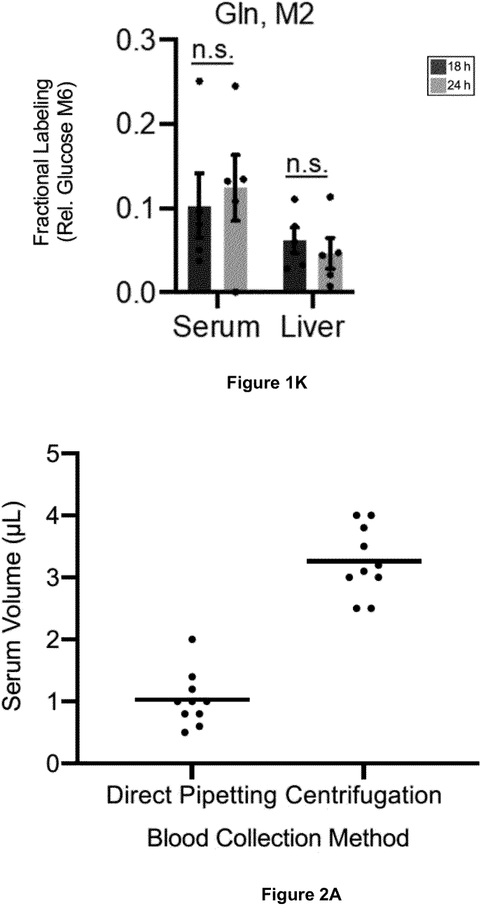

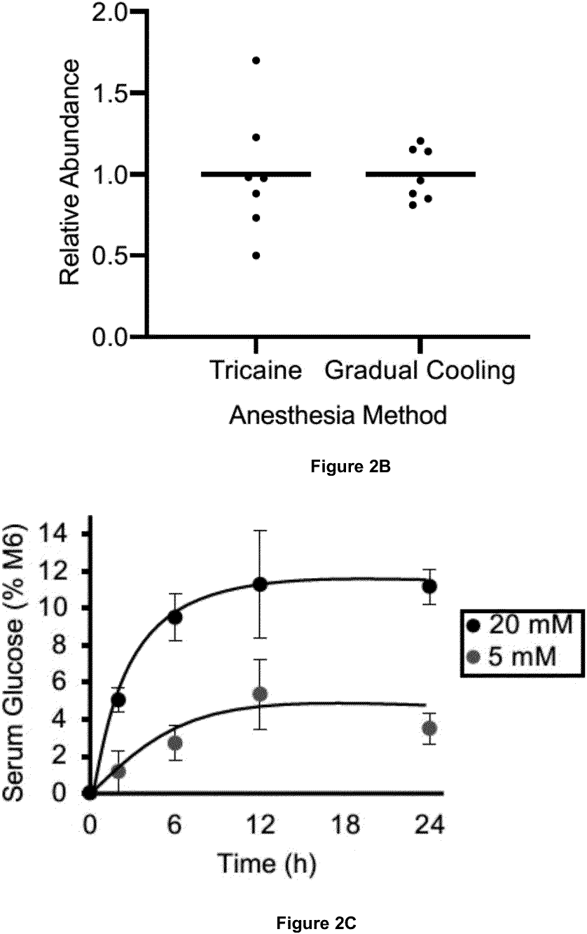

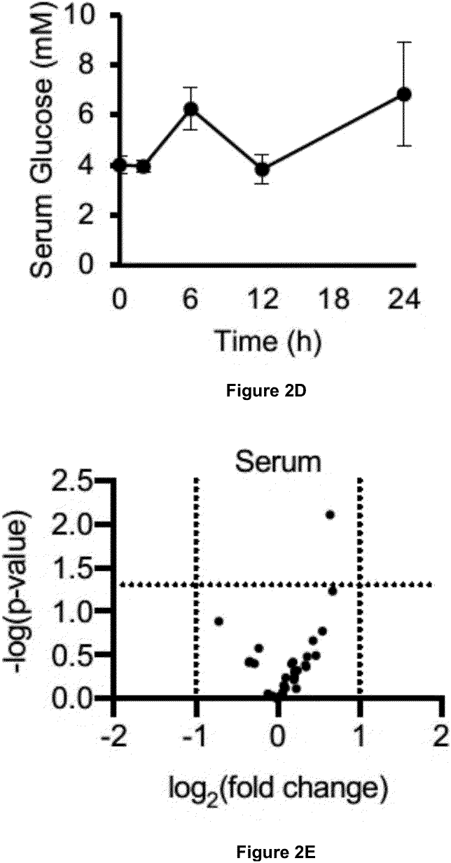

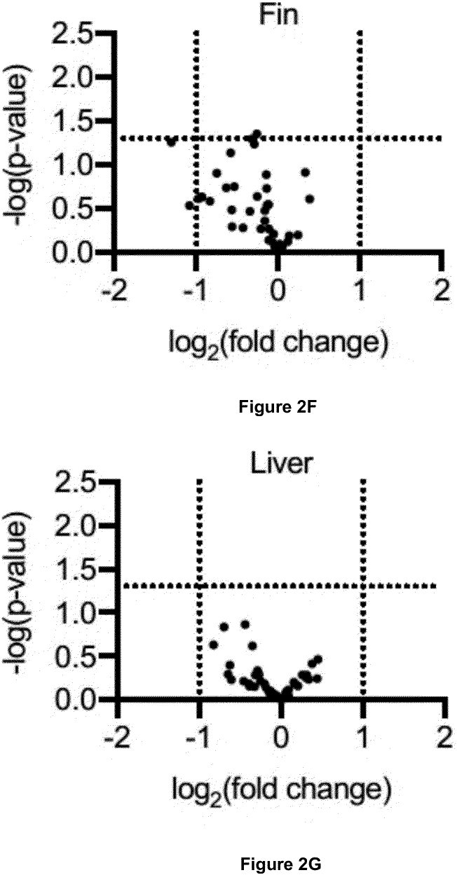

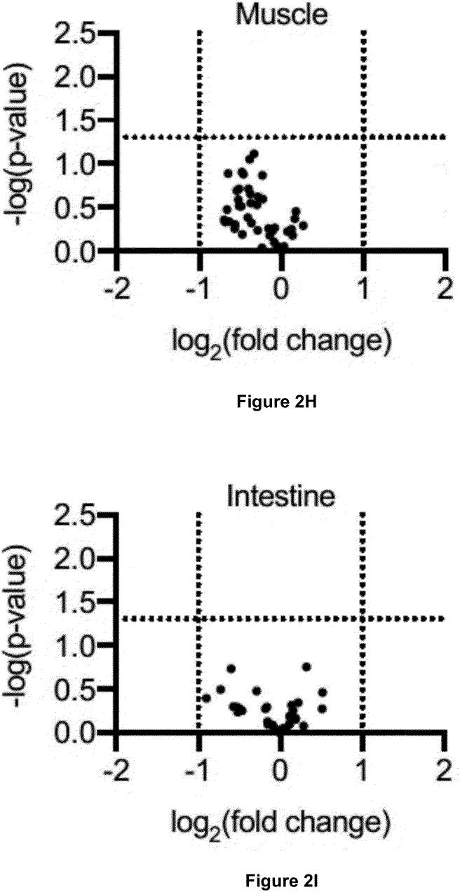

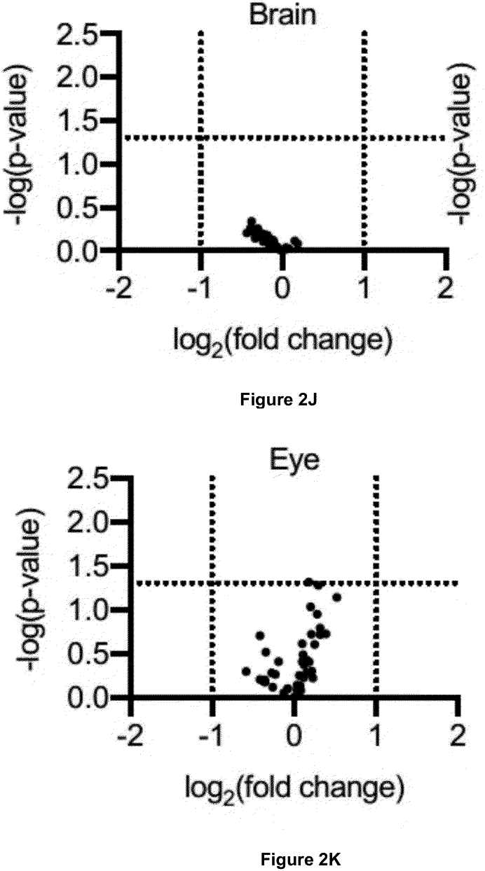

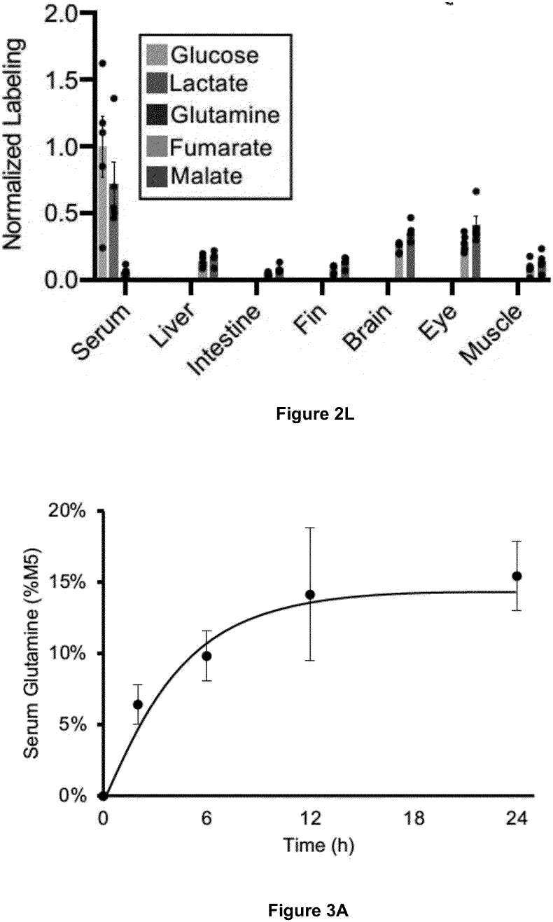

Given that isotope-tracer studies have not yet been performed in adult zebrafish, a workflow for the experiments were first established (FIG. 1A). The relatively small size of zebrafish complicates metabolomics analysis of serum and tissues from individual animals. To obtain sufficient serum for liquid chromatography/mass spectrometry (LC/MS) analysis from a single fish, a method that has been applied in proteomics was adapted. In brief, following caudal fin amputation, animals were subjected to low-speed centrifugation to collect blood (FIG. 2A). A nonchemical anesthesia was applied during the process, which was found led to more reliable measurements of metabolism compared to the commonly used tricaine methods (FIG. 2B).

Once an experimental workflow was in place to make metabolomics measurements from individual zebrafish, optimizing conditions for achieving isotopic steady state was performed. Although isotopic steady state is not required to perform isotope tracing, it can simplify interpretation of the data. Wild-type (WT) fish were fasted and then transferred to tank water containing [U-¹³C] glucose at a concentration of 5 - 20 mM. Within 24 hours, all of the concentrations tested led to isotopic steady state for circulating glucose (FIG. 1B and FIG. 2C). It was determined that the 10 mM condition was optimal because it maximized labeling of downstream metabolites while not causing any significant perturbations in circulating glucose levels (FIG. 1C and FIG. 2D). Moreover, after 24 hours, no significant alterations in metabolism were detected in animals transferred to tank water containing 10 mM glucose compared to control animals maintained under standard conditions (FIG. S1E-K). To ensure that isotopic steady state was achieved systemically after placing zebrafish in 10 mM [U-¹³C] glucose water for 24 hours, we also compared the fractional labeling of key central carbon metabolites in serum and liver with LC/MS. Fractional labeling is defined as the fraction of a given isotopologue for a metabolite normalized to the fraction of [U-¹³C] tracer in the circulation. Labeling of glycolytic metabolites, TCA cycle metabolites, and related amino acids were at isotopic steady state across the whole organism (FIGS. 1D-K).

Normalized labeling, defined as the percentage of ¹³C atoms in a metabolite normalized to the percentage of ¹³C atoms in a circulating tracer, has been used to assess the direct and indirect contributions of a tracer at isotopic steady state to a downstream metabolite in vivo. On the basis of normalized labeling, it was found that the glucose contribution to circulating lactate and glutamine in zebrafish (~70% and ~10%, respectively) was comparable to mice (~65% and ~10%, respectively) (FIG. 2L). Additionally, the contribution of glucose to the TCA cycle in most zebrafish tissues (e.g., ~15% in liver, ~12% in muscle, and ~7% in intestine) was similar to the corresponding mouse tissues (e.g., ~15% in liver, ~20% in muscle, and ~10% in intestine) (FIG. 2L). These findings indicated that the isotope-tracing results from glucose are largely conserved between WT zebrafish and mice.

### Example 2: Application to a Zebrafish Melanoma Model

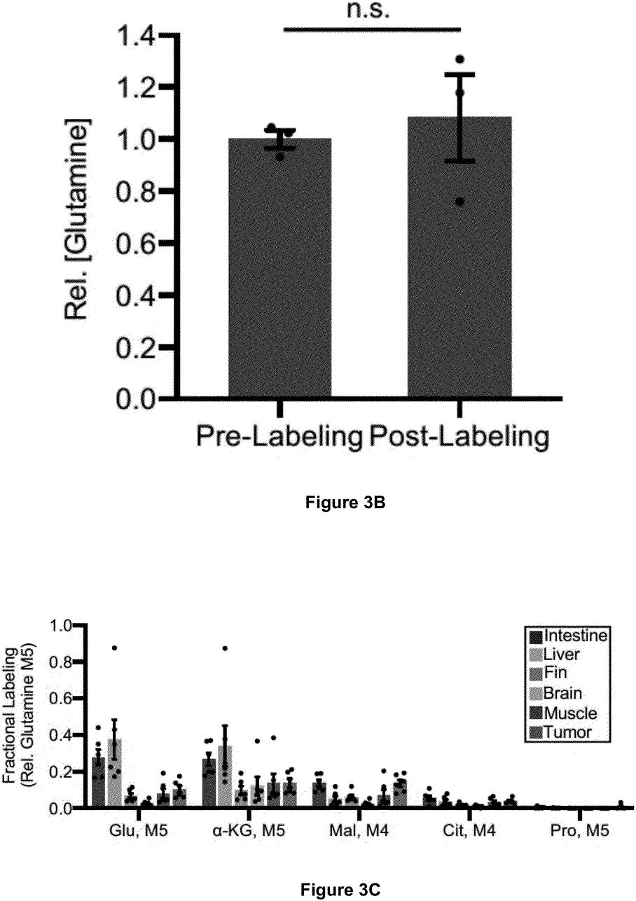

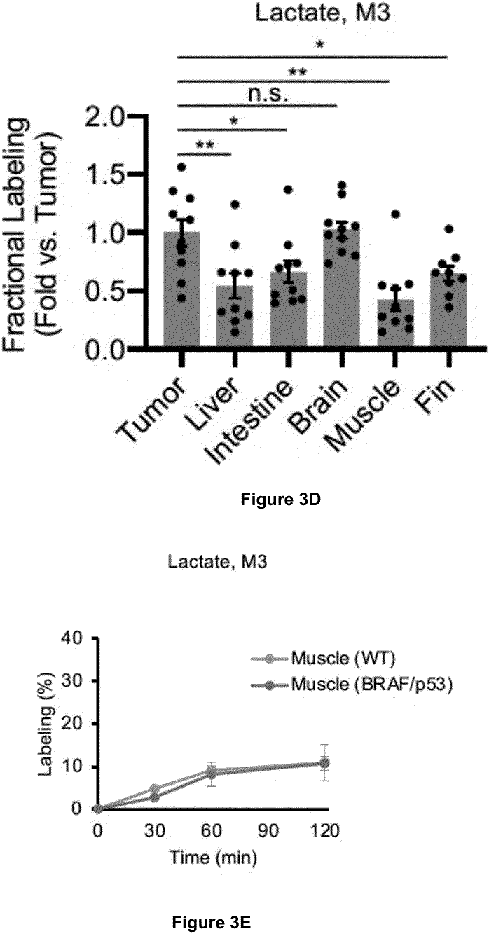

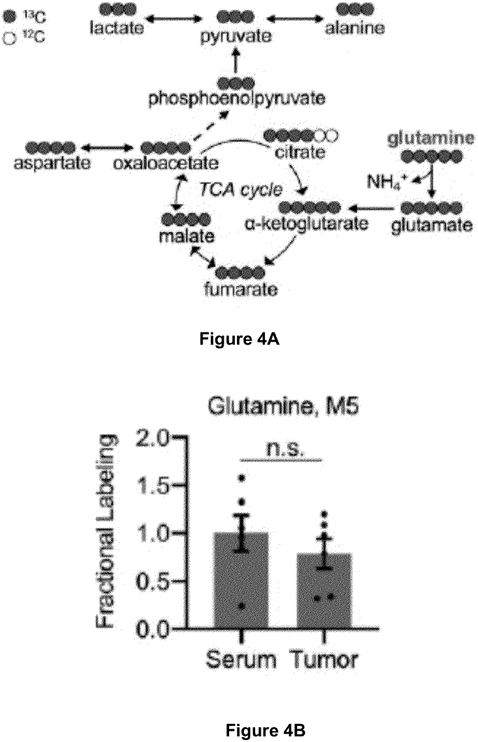

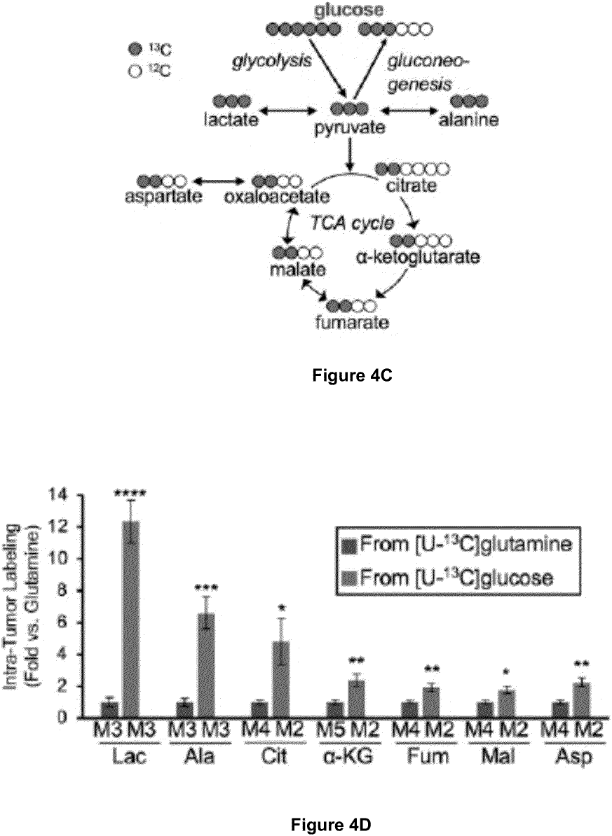

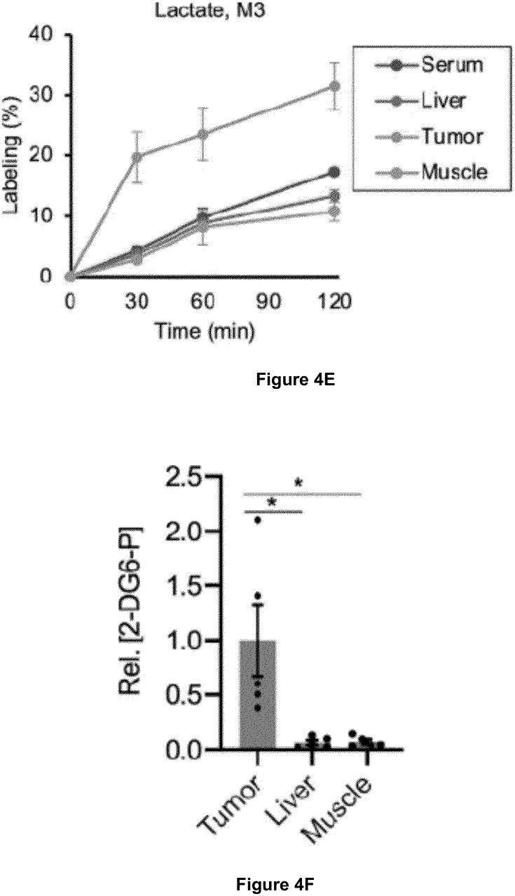

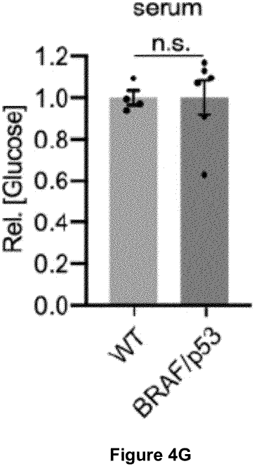

Having established the experimental workflow, the alterations in whole-body metabolism due to cancer was examined by using a zebrafish model of melanoma. Transgenic expression of the human oncogenic BRAF^{V600E} mutation under the control of the melanocyte mitfa promoter in a p53-deficient background leads to spontaneous melanoma development. Previous work has demonstrated that, in vitro, BRAF^{V600E}-driven melanoma cells are glutamine addicted, consuming up to 7-fold more glutamine than normal melanocytes. In cell-culture studies, BRAF^{V600E}-driven melanoma cells rely on glutamine as a precursor for TCA cycle intermediates and related metabolites such as proline and asparagine. Thus, it was hypothesized that a demand to sustain the TCA cycle with circulating glutamine carbon may represent a metabolic burden of melanoma on the organism. To test this, a BRAF/p53 (BRAF^{V600E}-mutant; p53-deficient) zebrafish was placed in 5 mM [U-¹³C] glutamine for 24 hours to achieve isotopic steady state. It was determined that these conditions were optimal for achieving steady-state labeling of serum glutamine without perturbation of circulating glutamine levels (FIGS. 3A-3B). Surprisingly, in contrast to previous in vitro studies, the disclosed in vivo data showed that glutamine contributed only minimally to the TCA cycle in melanoma (FIG. 4A and FIG. 3C). The fraction of labeled glutamine in melanoma matched that of the circulation, which indicated that low glutamine uptake was not the cause of the small contribution of glutamine to the TCA cycle in melanoma (FIG. 4B). Indeed, the contribution of glutamine to the TCA cycle in melanoma was comparable to fin tissue, which contains a high concentration of normal melanocytes (FIG. 3C). Liver and intestine, on the other hand, showed more extensive incorporation of glutamine carbon (FIG. 3C). These observations were consistent with other studies demonstrating that, for some cancers, glutamine utilization was decreased in vivo compared to in vitro. Given these results, it seemed unlikely that glutamine utilization by melanoma significantly impacts whole-body metabolism.

In addition to being glutamine addicted, BRAF^{V600E}-expressing melanoma cells are also known to increase their consumption of glucose relative to normal melanocytes in vitro. Therefore, examination of utilization of glucose in vivo by placing BRAF/p53 zebrafish in 10 mM [U-¹³C] glucose for 24 hours to achieve isotopic steady state was undertaken (FIG. 4C). It was observed that glucose contributed significantly more carbon than glutamine to glycolysis, the TCA cycle, and related amino acids (FIG. 4A, FIG. 4C and FIG. 4D). Additionally, all tissues with the exception of brain showed lower levels of labeling in glycolytic intermediates relative to tumor (FIG. 3D). To assess the flux of glycolysis in melanoma directly, 0.5 mg of [U-¹³C] glucose per gram of zebrafish via intraperitoneal injection was administered, and labeling in lactate was measured from various tissues as a function of time (FIG. 4E). The results demonstrated that the flux of glucose to lactate in melanoma was significantly higher than in other tissues, such as liver and muscle. From these data, it was predicted that tumors were consuming more glucose compared to other tissues. To assess glucose uptake, BRAF/p53 fish was placed in 1 mM 2-deoxyglucose (2-DG). Similar to glucose, 2-DG is taken up by glucose transporters and metabolized to 2-deoxyglucose 6-phosphate (2-DG6-P). Notably, 2-DG6-P cannot be further metabolized in cells, and it accumulates as a function of uptake rate. The relative concentrations of 2-DG in tissues was quantified and it was determined that tumors consumed 16-fold and 14-fold more glucose than the liver and muscle, respectively (FIG. 4F). In spite of this pathological demand for glucose introduced by the tumor, however, it was observed that circulating glucose levels were not different in BRAF/p53 fish as compared to WT (FIG. 4G). Additionally, glycolytic flux was not altered in muscle, showing that glucose was still being used as a fuel in non-malignant tissues (FIG. 3E).

### Example 3: Impact of Melanoma on Whole-Body Metabolism

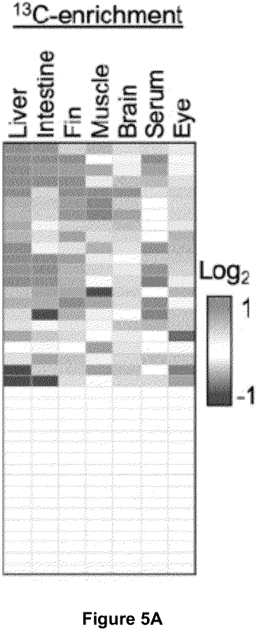

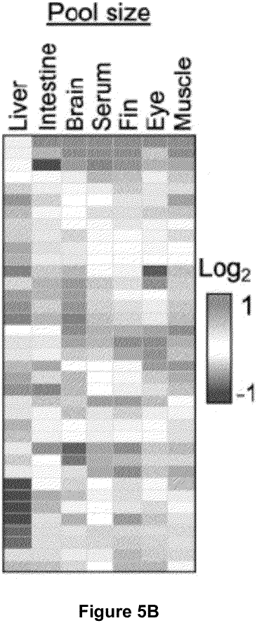

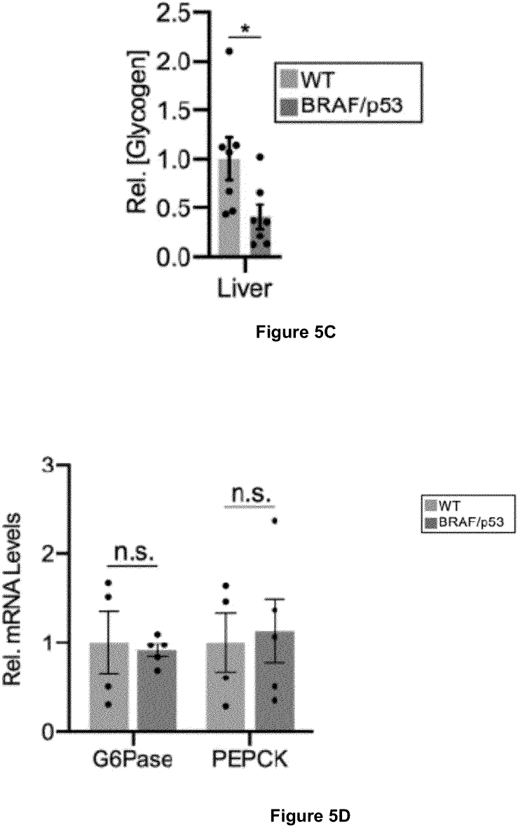

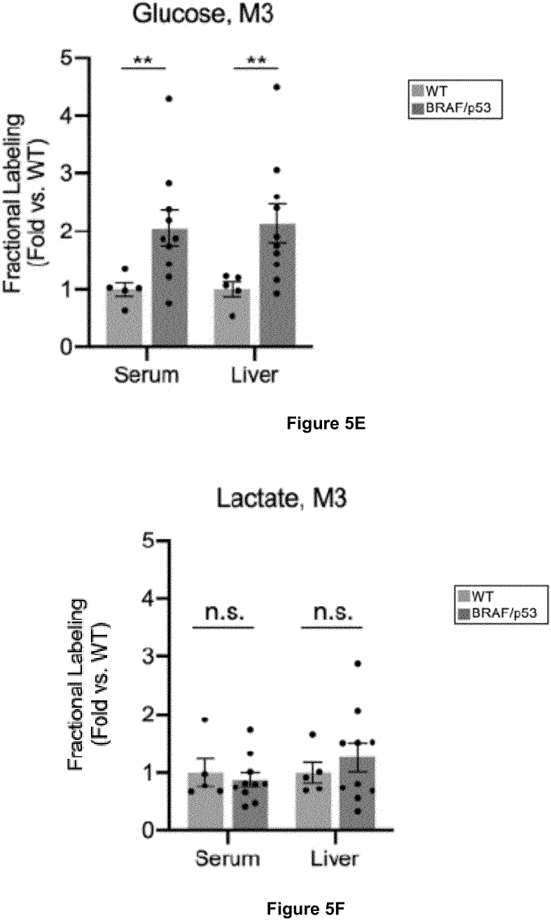

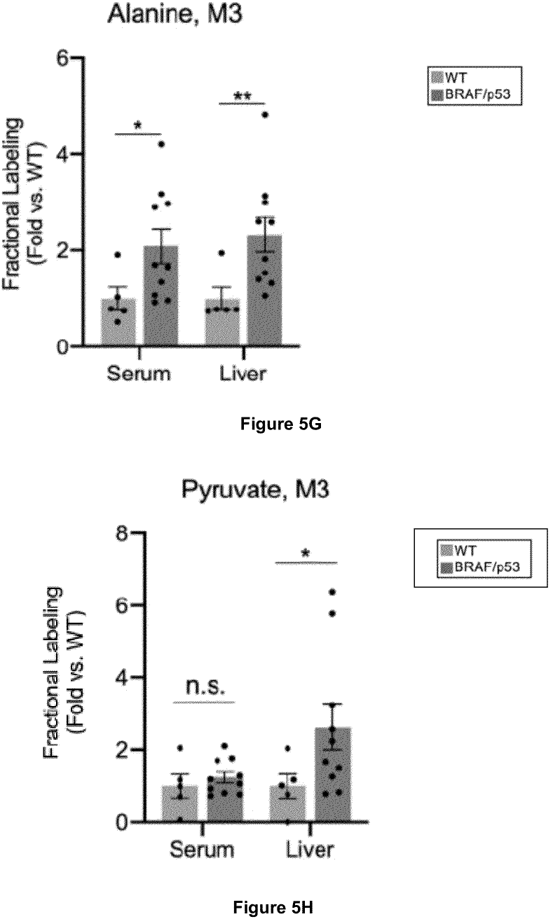

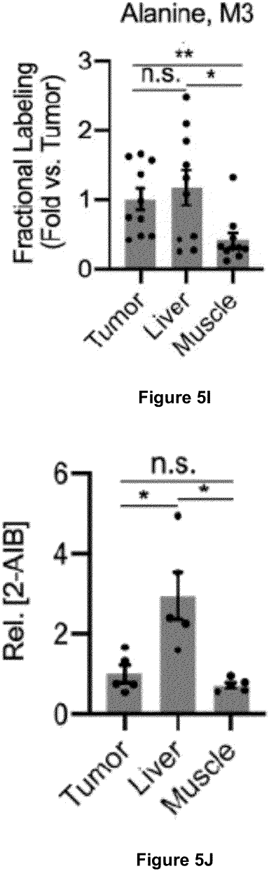

The findings disclosed above demonstrated that BRAF/p53 animals were able to maintain glucose homeostasis, even in the face of the metabolic burden imposed by the tumor. The data suggested that, while some tissues may compete with melanoma for glucose, other tissues were likely to reprogram their metabolism to complement the tumor. To evaluate the impact of melanoma on whole-body metabolism, LC/MS-based metabolomics was used to profile tissues from BRAF/p53 zebrafish after administration of an isotopically labeled glucose tracer. WT and melanoma-bearing zebrafish were placed in 10 mM [U-¹³C] glucose for 24 hours to achieve isotopic steady state, and ¹³C-enrichment and pool sizes of metabolites from WT were compared to BRAF/p53 animals (FIGS. 5A-5B). Seven different sample types were individually examined: liver, intestine, fin, muscle, brain, serum, and eye. Interestingly, metabolic dysregulation was observed across most tissues in BRAF/p53 fish, indicating that melanoma broadly impacted organismal glucose metabolism. The data provided herein is a resource to provide an integrative picture of alterations in whole-body metabolism that occured due to the presence of a tumor (FIGS. 5A-5B).

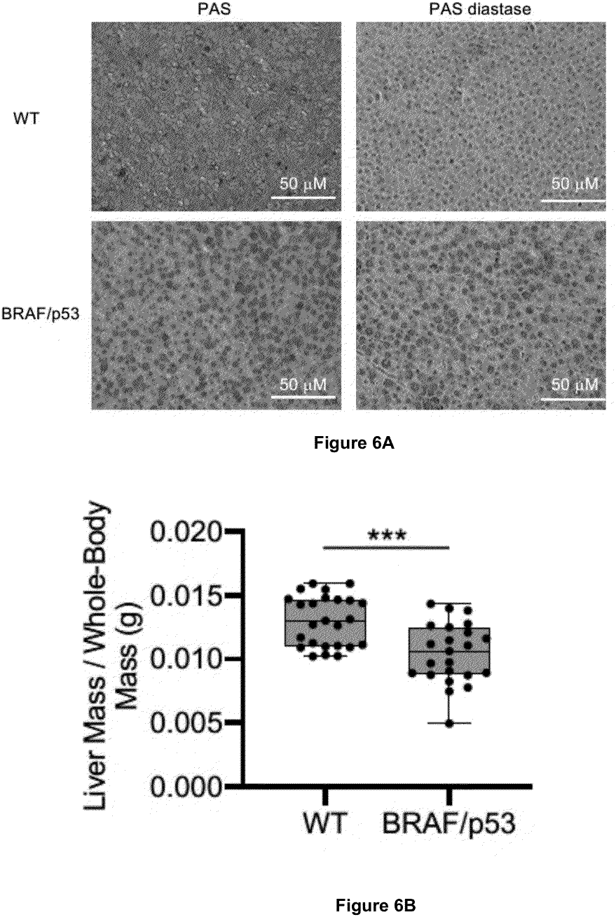

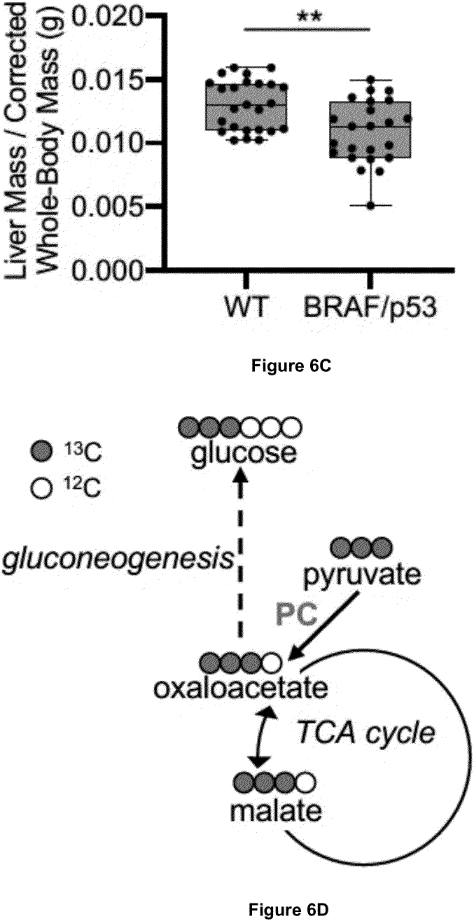

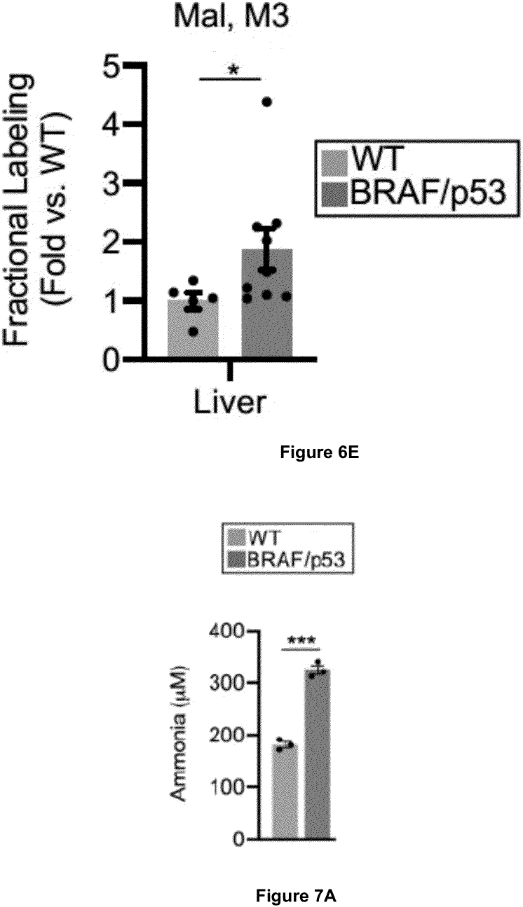

Given the magnitude of changes observed in the liver of tumor-bearing animals and the role of this organ in maintaining glucose homeostasis, study was further focused liver (FIGS. 5A-5B). It was hypothesized that the livers of BRAF/p53 fish might be increasing de novo glucose production as a compensatory measure in response to melanoma to maintain glucose homeostasis (FIG. 4G). Hepatic glucose production occurs primarily through two mechanisms: hydrolysis of glucose from glycogen (i.e., glycogenolysis) and production of new glucose molecules from non-carbohydrate precursors via gluconeogenesis. Glycogen accounts for as much as 15% of the total liver mass, and its degradation is the first mechanism activated to maintain systemic glucose levels. Consistent with increased glycogenolysis, it was found that the livers of BRAF/p53 fish had a significant reduction in glycogen content relative to the livers from WT fish as confirmed by a quantitative assay, Periodic Acid-Schiff staining, and decreased liver mass (FIG. 5C and FIGS. 6A-6C). Although the expression of genes involved in gluconeogenesis was unaltered in livers between WT and BRAF/p53 fish, data from [U-¹³C] glucose tracing revealed increased substrate flux through gluconeogenesis in disease animals (FIGS. 5D-5E). Specifically, an increase in M3 glucose labeling in the serum and livers of BRAF/p53 fish was observed, which is a signature of gluconeogenesis (FIG. 5E). Additionally, increased M3 malate labeling in the livers of fish with melanoma further supported higher gluconeogenic flux via pyruvate carboxylase (FIGS. 6D-6E).

### Example 4: Tumor-Liver Alanine Cycle

It was surmised that there were three candidates most likely to be used as gluconeogenic substrates by the liver of BRAF/p53 animals to support increased glucose production: lactate, alanine, and pyruvate. Lactate is the most abundant circulating three-carbon metabolite derived from glucose, is typically excreted at high concentrations by tumors, and is a well-established substrate for hepatic gluconeogenesis in healthy animals. M3 labeling of lactate, however, was not elevated in the circulation or liver of BRAF/p53 animals, despite it being readily produced by tumors (FIG. 5F). Thus, it seemed unlikely that lactate was used as a substrate to support increased gluconeogenesis in fish with melanoma. In contrast, M3 labeling of alanine was significantly increased in both the circulation and the liver of disease animals (FIG. 5G). Furthermore, M3 labeling of pyruvate was elevated in the livers, but not serum of BRAF/p53 fish (FIG. 5H). These data was consistent with the liver taking up more alanine from the circulation and transforming it into pyruvate within the liver to use as a substrate for gluconeogenesis (FIG. 3C, FIG. 5G, and FIG. 5H).

Conventionally, muscle is thought of as the major source of alanine production and participates in inter-organ alanine exchange with liver. Notably, in tumor-bearing animals, the data indicated a different paradigm of metabolite exchange where the main source of alanine for gluconeogenesis was melanoma rather than muscle. Two observations supported this observation. First, M3 labeling of alanine was over 2-fold higher in tumor relative to muscle (FIG. 51). Second, the relative labeling of M3 alanine was not statistically different between melanoma and liver (FIG. 51). To confirm tissue uptake of alanine and the directionality of alanine transfer, BRAF/p53 zebrafish was transferred to tank water containing 0.5 mM of the non-metabolizable alanine analog 2-aminoisobutyric acid (2-AIB). Measurement of 2-AIB levels in tissue provides a proxy for alanine uptake. The concentrations of 2-AIB in liver, tumor, and muscle were compared and found that relative alanine uptake in liver was ~3-fold higher than in melanoma. In contrast, the amount of alanine taken up by melanoma was similar to that of muscle (FIG. 5J). Taken together, the disclosed data showed that melanoma produced more alanine than muscle and take up less alanine than liver, thereby supporting that melanoma-derived alanine contributed to a non-conventional tumor-liver alanine cycle to replenish circulating glucose in animals with tumors.

### Example 5: The Alanine Cycle Removes Excess Nitrogen from Catabolism of Branched-Chain Amino Acids

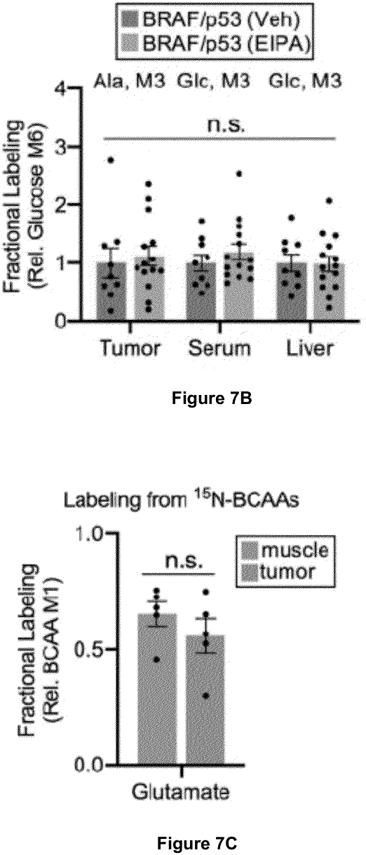

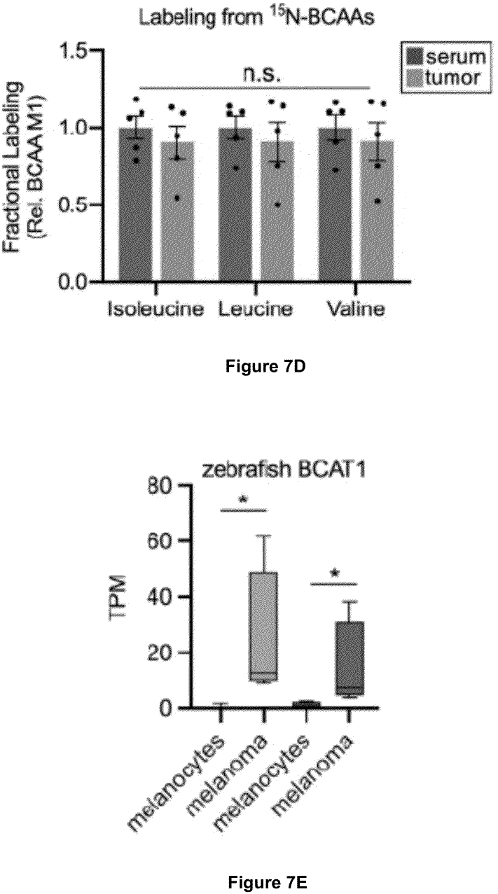

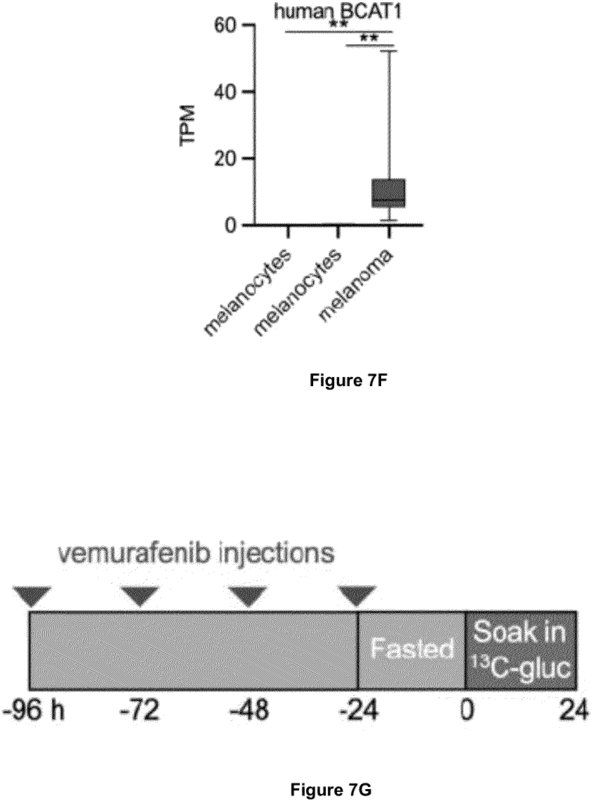

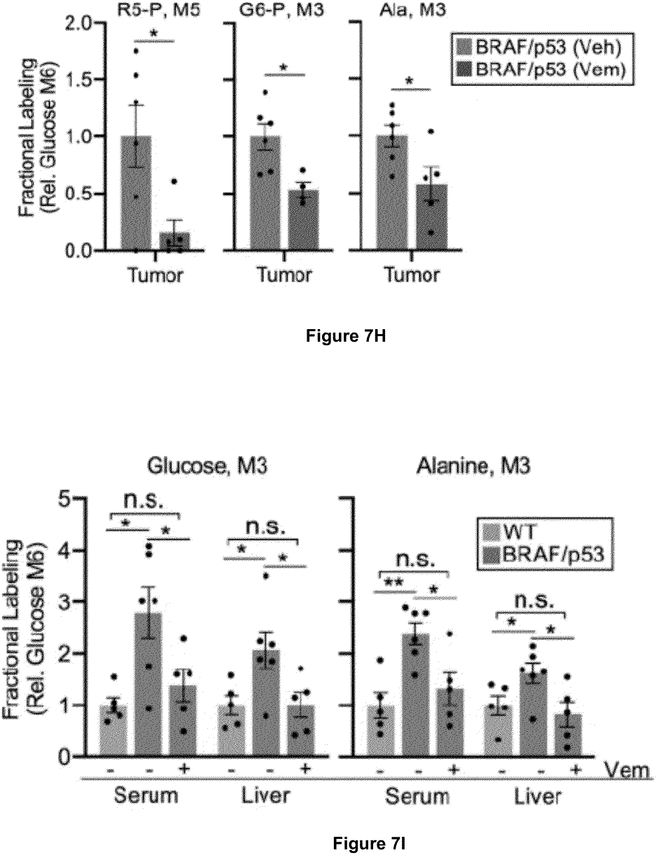

In vertebrates, the liver plays a major role in maintaining systemic nitrogen homeostasis. In contrast to urea excretion in mammals, however, the primary process by which zebrafish remove nitrogenous waste is ammonotelism, where liver-produced ammonia from amino acid catabolism is excreted directly through the gills. This waste-removal mechanism (and other processes like it) exemplify another advantage of using zebrafish in metabolism research: tank water can be sampled repeatedly and non-invasively to measure excretory molecules derived from metabolic processes. As such, it was examined whether tumor-liver alanine cycling in BRAF/p53 fish was associated with aberrant ammonia excretion compared to WT fish. BRAF/p53 and WT fish were housed separately in fresh water, and ammonia levels were measured after 24 hours. In agreement with pathological alanine cycling, animals with tumors excreted nearly twice as much ammonia as WT fish (FIG. 7A).

Next, the source of the nitrogen being excreted from tumors was determined. The uptake of circulating protein through macropinocytosis, was considered initially, a process known to provide an important source of nutrients for pancreatic tumors. It was reasoned that oxidation of the carbon skeletons of protein-derived amino acids could lead to an excess of intracellular nitrogen that needed to be removed. To assess the potential contribution of protein uptake to nitrogen balance, tumor-bearing animals were treated with the macropinocytosis inhibitor ethyl-iso-propyl-amiloride (EIPA) and then alanine cycling was examined. Even at high concentrations, EIPA did not affect alanine labeling in the tumor or hepatic gluconeogenesis (as reflected by M3 labeling of glucose in the liver and serum). These data indicated that macropinocytosis does not provide a significant contribution to the nitrogen balance of tumors in the disclosed model (FIG. 7B).

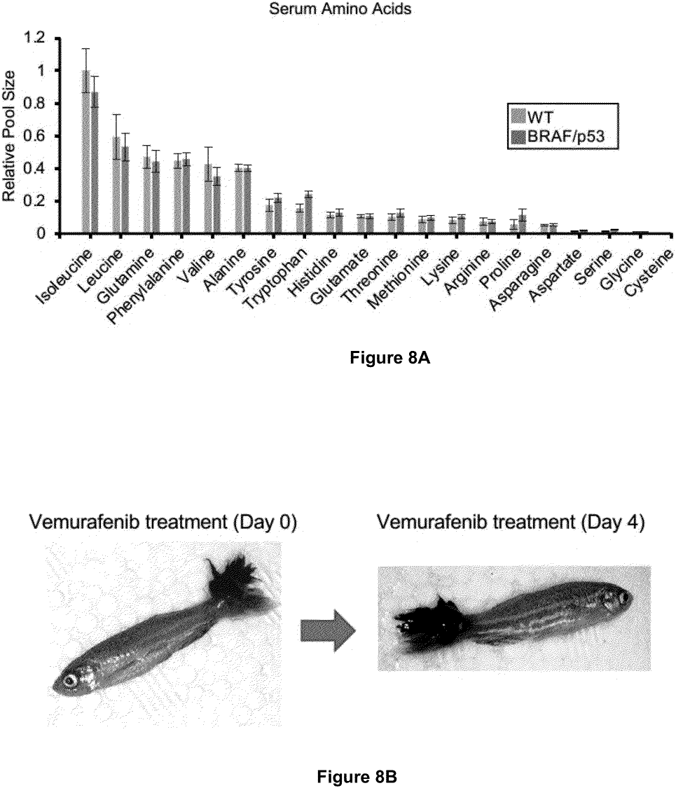

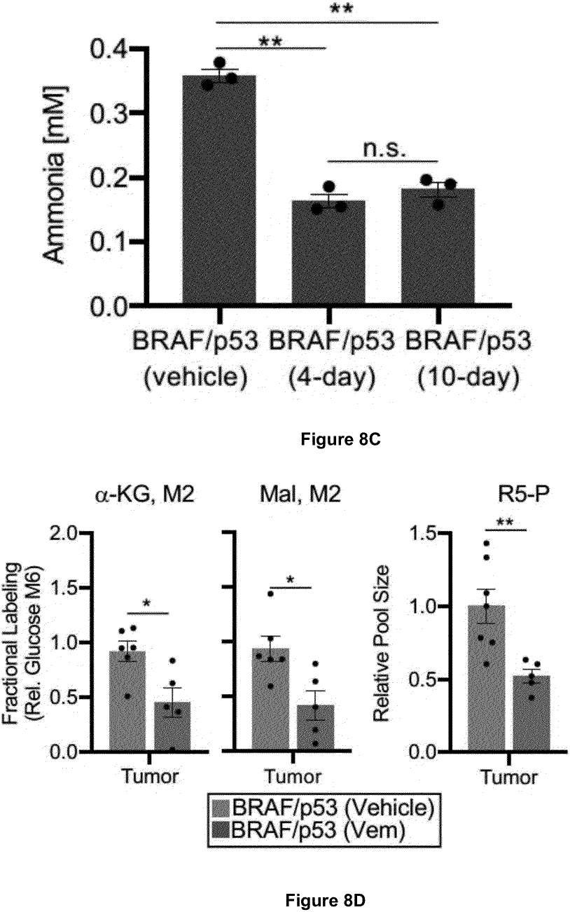

As an alternative source of nitrogen, a second possibility was considered, that tumors might take up free amino acids directly from the circulation. It was surmised that the most abundant amino acids in the serum of zebrafish would be likely to contribute the most nitrogen. With LC/MS-based metabolomics, it was determined that the amino acids at the highest concentration in the serum of both WT and BRAF/p53 zebrafish were isoleucine, leucine, phenylalanine, glutamine, alanine, and valine (FIG. 8A). The data disclosed above showed that the contribution of glutamine to tumor metabolism is minimal (FIG. 4D). Thus, the branched chain amino acids (BCAAs) were examined. BRAF/p53 zebrafish was placed in water containing 2 mM [¹⁵N] isoleucine, 2 mM [¹⁵N] leucine, and 2 mM [¹⁵N] valine for 24 hours. The first step in BCAA degradation is transfer of the α-amino group to a-ketoglutarate to form glutamate through the activity of branched-chain amino acid aminotransferase (BCAT). The nitrogen can then be transferred to pyruvate by alanine aminotransferase (ALT), producing alanine for removal. Interestingly, free BCAAs taken up from the circulation contributed nearly 60% of the total nitrogen to glutamate in the tumor, suggesting that these amino acids were the largest donor of nitrogen to be removed from the tumor (FIG. 7C). Additionally, the amount of glutamate labeled by [¹⁵N] BCAAs was not significantly different than in muscle (FIG. 7C). Muscle is known to be a main site of BCAA catabolism, which results in a surplus of nitrogen that is released into the circulation as alanine. These data further suggested that melanoma exhibited similar metabolism to muscle, taking up BCAAs and excreting their nitrogen via alanine. As further evidence that macropinocytosis was not a major contributor to the nitrogen pool in melanoma, the labeling of BCAAs in the tumor was not significantly different than in serum (FIG. 7D). This result was consistent with labeled BCAAs taken up by the tumor not being diluted by unlabeled BCAAs that resulted from the degradation of intact proteins.

To confirm that melanoma oncogenesis activates BCAA degradation, expression of branched chain amino acid transferase 1 (BCAT1) was compared in melanoma cells to normal melanocytes. It was found that expression of BCAT1 in zebrafish melanocytes was near zero. In contrast, expression was significantly up-regulated in melanoma cells from BRAF/p53 tumors (FIG. 7E). The BCAT1 expression was further evaluated in tumors obtained from a cohort of 18 patients with advanced melanoma treated at Washington University School of Medicine. Similar to what was observed in zebrafish, relative to healthy human melanocytes, BCAT1 was highly expressed in melanoma (FIG. 7F). These results suggested that BCAA catabolism was activated in melanoma from both a zebrafish model of disease and in human tumors.

### Example 6: Inhibiting BRAF^{V600E} with Vemurafenib Reduces Tumor-Liver Alanine Cycling

Next, it was considered whether a selective inhibitor of BRAF^{V600E}, vemurafenib, affects tumor-liver alanine cycling. Vemurafenib is a clinically-approved chemotherapy for melanoma expressing the BRAF^{V600E} mutation that has minimal toxicity and high tolerability in humans, mice, and zebrafish. On the basis of efficacy and toxicity profiles previously reported for zebrafish and murine melanoma models, vemurafenib was administered at 100 mg/kg/day via intraperitoneal injection to BRAF/p53 zebrafish. After 4 days of consecutive vemurafenib or DMSO vehicle treatment, animals were transferred to new tanks containing 10 mM [U-¹³C] glucose for 24 hours prior to quantifying ammonia excretion and performing metabolomics (FIG. 7G). After 4 days of drug treatment, no significant reduction in tumor size had occurred but a robust metabolic response was observed (FIG. 8B). Compared to vehicle-treated BRAF/p53 fish, animals given vemurafenib for 4 days: (i) excreted significantly less ammonia (FIG. 8C); (ii) incorporated less ¹³C-label into central carbon metabolites in the tumor, with M3 labeling of alanine reduced by ~2-fold (FIG. 7H and FIG. 8D); and (iii) showed a reduction in the pool size and ¹³C-labeling of ribose 5-phosphate (R5-P), a nucleotide precursor whose production is upregulated in proliferating cells (FIG. 7H and FIG. 4D). Moreover, BRAF/p53 animals treated with vemurafenib for 4 days had levels of gluconeogenesis that were comparable to WT zebrafish, as indicated by M3 labeling in hepatic and circulating glucose (FIG. 71). In addition, no significant differences were observed in hepatic and circulating M3 alanine between BRAF/p53 animals treated with vemurafenib for 4 days and WT fish (FIG. 71). Collectively, these data show that inhibiting BRAF^{V600E} led to the loss of alanine cycling between tumor and liver.

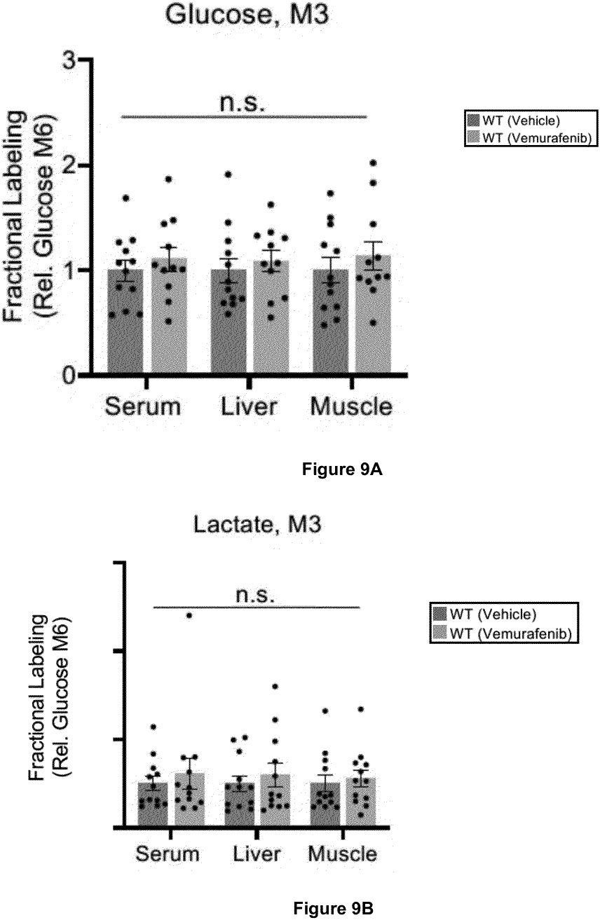

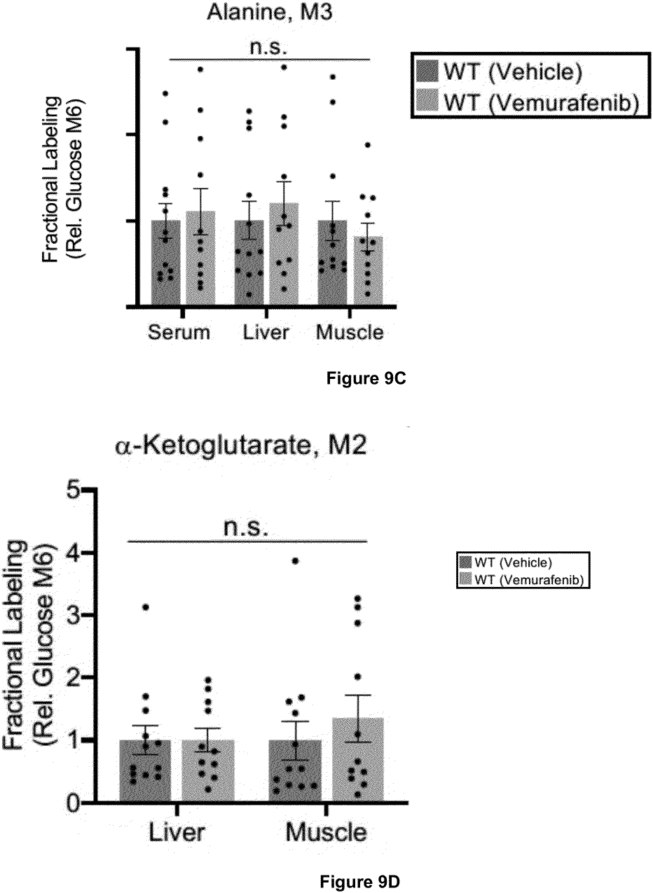

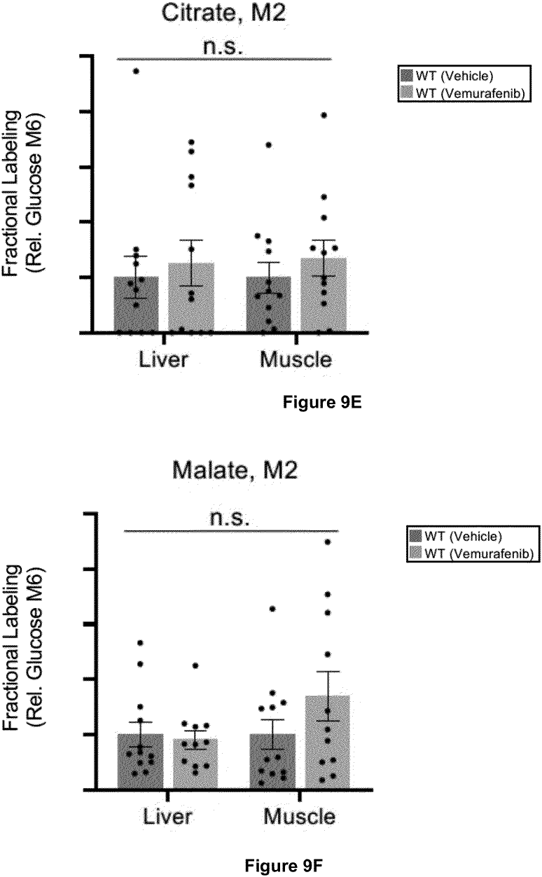

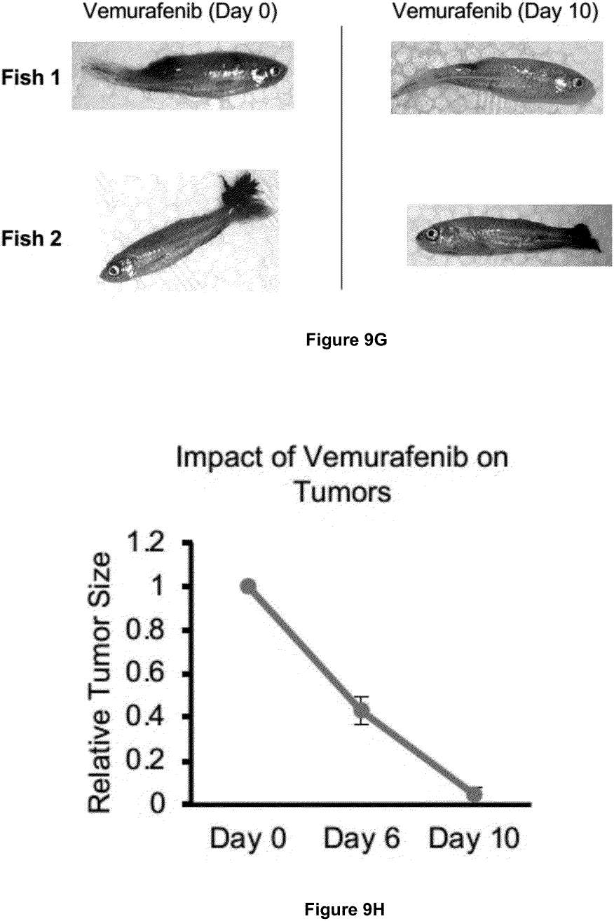

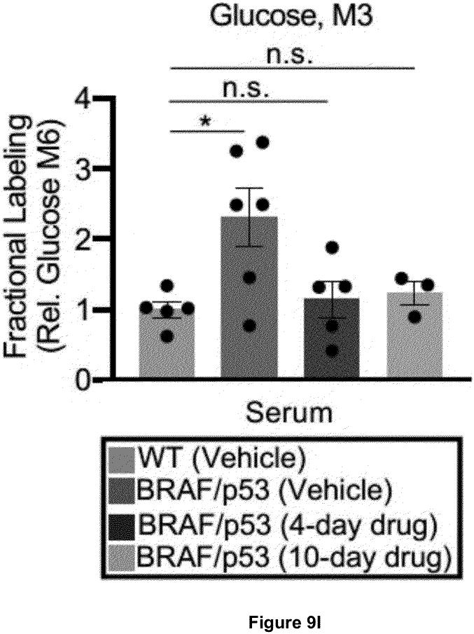

To better understand the effects of vemurafenib, two additional experiments were performed. First, WT zebrafish administered vehicle and WT zebrafish administered vemurafenib at 100 mg/kg/day were compared for 4 consecutive days. Following vehicle or vemurafenib treatment, the animals were transferred to tank water containing [U-¹³C] glucose for 24 hours. No differences in ¹³C-labeling were found in metabolites from the circulation, liver, or muscle (FIGS. 9A-9F). These data indicated that vemurafenib did not affect alanine cycling in the absence of BRAF^{V600E}. Second, the same experiments outlined above were performed with vemurafenib, but the drug was administered for 10 consecutive days at 100 mg/kg/day. After 10 days of treating BRAF/p53 zebrafish with vemurafenib, tumors were reduced in size by >95% (FIGS. 9G-9H). Yet, ammonia excretion after 10 days of vemurafenib treatment was not significantly different from ammonia excretion after 4 days of vemurafenib treatment (FIG. 8C). Isotope tracing data from animals treated with vemurafenib for 4 and 10 days were also comparable (FIG. 9I). These results indicated that alanine cycling is not reduced in BRAF/p53 zebrafish due to tumor regression.

### Example 7: Inhibiting ALT Reduces BCAA Catabolism, Alanine Cycling, and Tumor Volume

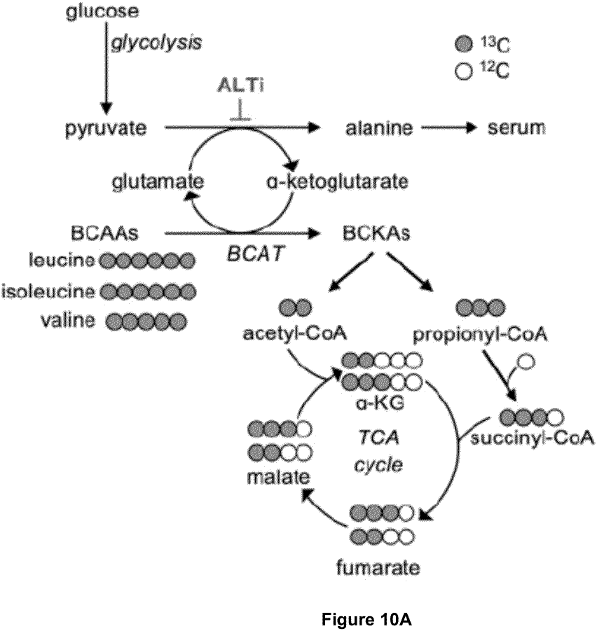

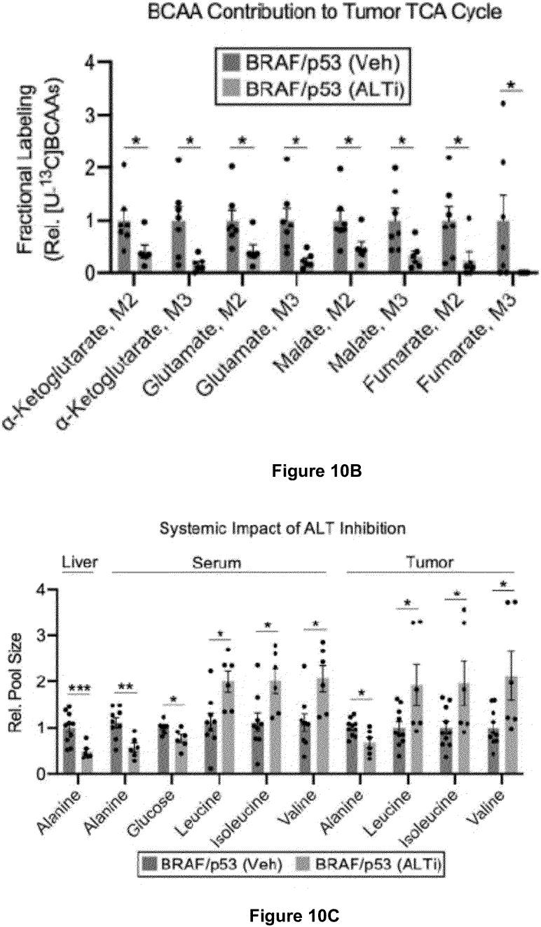

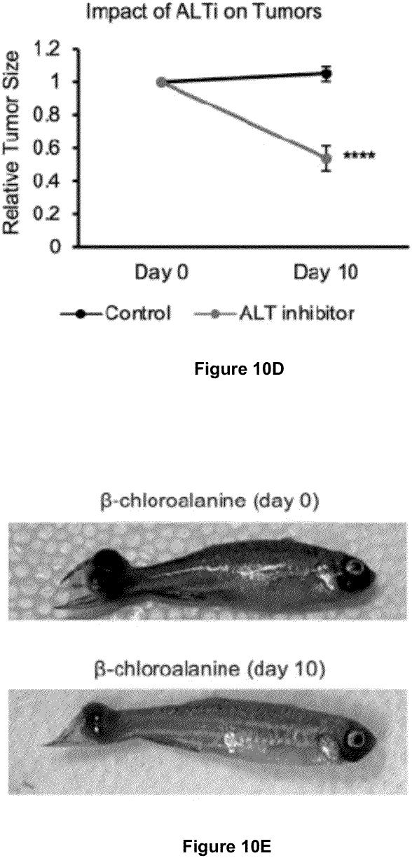

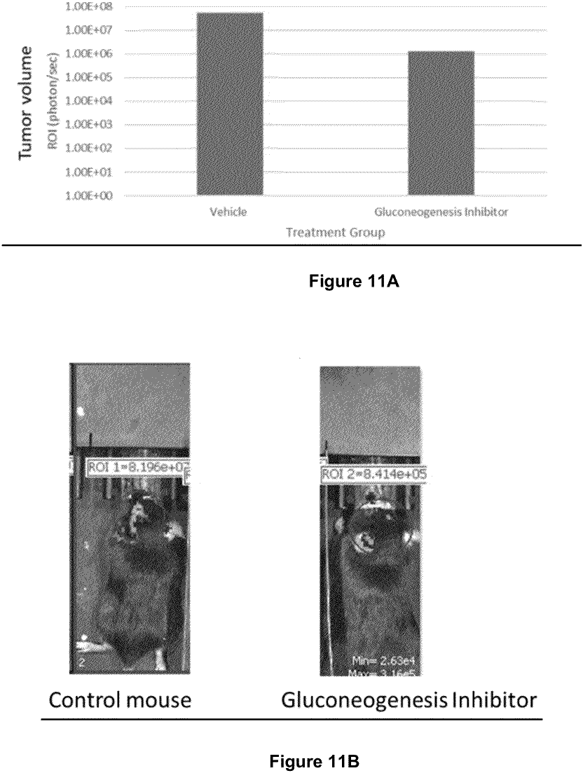

The observations disclosed above suggested that activating BCAA degradation in melanoma results in excess nitrogen, which is released from the tumor via alanine and then transported to the liver for removal as ammonia. Thus, evaluation was done whether inhibiting alanine production in melanoma influenced BCAA catabolism and subsequent tumor growth. First, melanoma-bearing zebrafish was treated with an ALT inhibitor (β-chloroalanine) for 10 days. Next, the animals were transferred to tank water containing [U-¹³C] BCAA for 24 hours. Given that BCAAs enter the TCA cycle as acetyl-CoA and propionyl-CoA following the removal of their α-amino group, which ultimately is transferred to pyruvate via ALT (FIG. 10A), the contribution that BCAAs made to the TCA cycle in tumors were assessed. Inhibiting ALT with β-chloroalanine significantly reduced the contribution that [U-¹³C] BCAAs made to the TCA cycle (FIG. 10B). Furthermore, ALT inhibition led to an accumulation of BCAAs in serum, an accumulation of BCAAs in tumor, decreased levels of circulating glucose, and decreased levels of alanine in tumor, serum, and liver (FIG. 10C). Reducing oxidation of BCAAs and alanine production in melanoma also correlated with a 50% reduction in tumor size (FIGS. 10D-10E). Together, these results indicated that BCAA catabolism contributes to tumor growth and that removal of nitrogen from the tumor via alanine was required for efficient use of these substrates by melanoma.

### Example 8: Treatment with Gluconeogenesis Inhibitor Reduces Tumor Volume in Mouse

To test whether gluconeogenesis inhibitor has an effect on tumor in mouse, glioblastoma was induced in mice through intracranial injection of 50,000 luciferase expressing GL261 cells. The mice were treated either with vehicle or 50 mg/kg of a gluconeogenesis inhibitor delivered via intraperitoneal injection, and average tumor volume was measured using IVIS imaging. The mouse treated with gluconeogenesis inhibitor exhibited a reduction in tumor volume compared to control mouse.

### SUMMARY

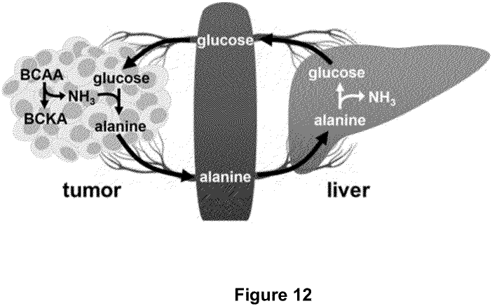

In summary, the work presented here established the adult zebrafish as a well-suited model organism to study whole-body physiology with isotope-tracer analysis and metabolomics. The experimental procedures disclosed herein can be easily adapted to study any zebrafish model of disease. Using a BRAF/p53 model of melanoma, it was shown that the presence of a tumor leads to systemic alterations in metabolism (FIG. 12). In particular, the results demonstrated that the liver uses tumor-excreted alanine as a substrate for gluconeogenesis. The process maintained circulating glucose levels and helped support the high glycolytic demands of the tumor. In melanoma, it demonstrated that the cycle supported the removal of excess nitrogen from the tumor obtained from increased catabolism of BCAAs and that blocking the cycle with an inhibitor of ALT could be an attractive treatment. The studies disclosed herein showed the importance of metabolic cooperation between the tumor and otherwise healthy tissues in an organism, providing an example of how tumors could exploit the normal physiology of the host for its own benefits. Characterizing such interactions has the potential to reveal new metabolic opportunities for therapeutic intervention associated with distal, non-malignant tissues rather than the tumor itself.

What is claimed is:

1. A composition for treatment of cancer in a subject in need thereof, the composition comprising an inhibitor of alanine aminotransferase and at least a pharmaceutically acceptable excipient.

2. The composition of claim 1, wherein the inhibitor of alanine aminotransferase is β-chloro-L-alanine or a pharmaceutically acceptable salt, derivative, variant or a functional analog thereof.

3. The composition of claim 1, wherein the pharmaceutically acceptable excipient comprises a binder, a filler, a disintegrant, a lubricant, a glidant, a salt, a polymer, buffering agent, solvent, or a combination thereof.

4. The composition of claim 1, wherein the cancer is a melanoma.

5. The composition of claim 1, wherein the subject is a vertebrate.

6. The composition of claim 5, wherein the subject is a human.

7. The composition of claim 2, wherein the composition is in a unit dose form wherein the β-Chloro-L-alanine or a pharmaceutically acceptable salt, derivative or analog thereof is present in an amount selected from a range of 10 mg - 2 grams.

8. The composition of claim 2, wherein the composition is in the form of a tablet, a powder, a capsule, a liquid, or injectable.

9. The composition of claim 1, wherein administration of the composition in the subject disrupts the tumor-liver alanine cycle.

10. A method for treatment of a tumor or a cancer in a subject in need thereof, the method comprising administering to the subject an effective amount of a composition comprising an inhibitor of alanine aminotransferase and at least a pharmaceutically acceptable excipient.

11. The method of claim 10, wherein the inhibitor of alanine aminotransferase is a β-chloro-L-alanine or a pharmaceutically acceptable salt, derivative or a functional analog thereof.

12. The method of claim 10, wherein the administration of the inhibitor disrupts the tumor-liver alanine cycle.

13. The method of claim 10, wherein the administration of the inhibitor results in a reduction of the dimensions of the tumor by at least about 10% to about 50%.

14. The method of claim 11, wherein the β-chloro-L-alanine or a pharmaceutically acceptable salt, derivative or analog thereof is administered by a mode selected from parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, and transdermal.

15. The method of claim 12, wherein the β-chloro-L-alanine is administered at a unit dose amount selected from a range of 10 mg - 2 grams.

16. The method of claim 10, wherein the cancer is a melanoma.

17. The method of claim 10, wherein the subject is a vertebrate.

18. The method of claim 17, wherein the subject is a human.

19. A method of analysis of tumor metabolism in a laboratory animal tumor model, the method comprising:
a) continuous administrating of an isotope tracers over a period of time through direct ingestion of fluids;
b) obtaining a blood sample from the laboratory animal after step a);
c) conducting a metabolomics analysis using an LC/MS;
d) comparing the hepatic gluconeogenesis flux in the laboratory animal tumor model with the hepatic gluconeogenesis flux of a healthy animal model without tumor.

20. The method of claim 19, wherein the laboratory animal is a zebra fish.
